(19) United States
(12) Patent Application Publication
(10) Pub. No.: US 2023/0093832 A1
(43) Pub. Date: Mar. 30, 2023

(54) COMPOSITIONS AND METHODS FOR CONTROLLED RELEASE OF TARGET AGENT

(71) Applicant: THE HONG KONG UNIVERSITY OF SCIENCE AND TECHNOLOGY, Hong Kong (CN)

(72) Inventors: Ying CHAU, Hong Kong (CN); Chi Ming Laurence LAU, Hong Kong (CN); Yu YU, Shenzhen (CN); Zhexun SUN, Shenzhen (CN)

(73) Assignee: THE HONG KONG UNIVERSITY OF SCIENCE AND TECHNOLOGY, Hong Kong (CN)

(21) Appl. No.: 17/792,968

(22) PCT Filed: Jan. 13, 2021
(86) PCT No.: PCT/CN2021/071567

§ 371 Date: Jul. 14, 2022

(30) Foreign Application Priority Data
Jan. 16, 2020 (CN) PCT/CN2020/072509

(51) Int. Cl. A61K 9/06; A61K 47/36

(52) U.S. Cl. CPC A61K 9/06; A61K 47/36

(57) ABSTRACT

Provided are compositions and methods for controlled release of macromolecules (such as proteins and polypeptides). The composition comprises at least a first hydrogel forming polymer and at least a second hydrogel forming polymer. Also provided are methods for preparing and using the composition.

### BACKGROUND OF THE INVENTION

Hydrogels are three-dimensional network of polymers with water or other materials (e.g., macromolecules) entrapped within the polymer network. The size of the three-dimensional voids created by the polymer matrix is called “mesh size” or ξ In theory, by controlling the mesh size to be similar to the macromolecules (e.g., such as proteins, polypeptides and aptamers), the macromolecule can be controlled.

However, due to polydisperse nature of the precursor polymers, the random process of the crosslinking step, and the existence of the cargo proteins which interfere the construction of the polymer network, precise controlling the mesh size and its distribution is difficult. Therefore, the mesh-size-control-based depot system always yield unsatisfactory release profiles. The macromolecules located in the looser meshes can be released, while those in tighter meshes are hardly diffusible and can be considered as physically immobilized. If the crosslinks of the polymer matrix are degradable, the tighter mesh size can enlarge and the portion of macromolecules which were trapped could be liberated. Therefore, coupling the release of the laden molecules to the degradation of the depot meshwork can be an effective strategy to better control the drug release behaviors.

An issue for the encapsulation of macromolecules in hydrogel is that the macromolecules were often covalently bound to the polymer network, and thus are not free proteins.

Accordingly, a versatile, effective, and/or customizable approach is highly needed to achieve sustained release of macromolecules, such as proteins, polypeptides and aptamers.

### SUMMARY OF THE INVENTION

The present disclosure provides compositions and methods for controlled release of macromolecules (such as proteins and polypeptides). With the systems and methods of the present disclosure (e.g., the mass ratio between the first modification and the second modification is less than about 1), undesirable covalent binding between macromolecules and polymer can be eliminate. For example, at least about 20% (e.g., at least about 20%, at least about 30%, at least about 40%, at least about 50%, at least about 60%, at least about 70%, at least about 80%, at least about 90%, at least about 91%, at least about 92%, at least about 93%, at least about 94%, at least about 96%, at least about 98%, at least about 99%, or more) portion of macromolecule are free in the hydrogel network. Besides, the compositions and methods of the present disclosure are capable of adjusting a suitable hydrogel environment (e.g., hardness, gel time, swelling rate, etc.). The macromolecules may be retained within a structure (e.g., hydrogel) formed by polymers, which may be degraded (e.g., through hydrolytic cleavage) during an extended period of time (e.g., over days, weeks, or even months). The degradation may occur under physiological conditions. The polymers as well as its degradation products may be biocompatible. The polymer structure (e.g., hydrogel) may be formed in situ, for example, a composition (e.g., a liquid formulation) capable of forming the polymer structure (e.g., hydrogel) may be introduced (injected) into a tissue, and then, the polymer structure (e.g., hydrogel) may be formed in situ within the tissue upon being introduced. The release of the target molecule from the hydrogel can be controlled.

In one aspect, the present disclosure provides a composition comprising at least a first hydrogel forming polymer and at least a second hydrogel forming polymer, said first hydrogel forming polymer is capable of reacting with said hydrogel forming second polymer to form said hydrogel, and said hydrogel is degradable and enables sustained release of a target agent, wherein said first hydrogel forming polymer comprises a first hydrogel forming polymer derivative, said first hydrogel forming polymer derivative comprises a first modification, and said first hydrogel forming polymer derivative is electrophilic, and said second hydrogel forming polymer comprises a second hydrogel forming polymer derivative, said second hydrogel forming polymer derivative comprises a second modification, and said second hydrogel forming polymer derivative is nucleophilic; and a mass ratio between said first hydrogel forming polymer and said second hydrogel forming polymer is less than 1.

In some embodiments, said first modification is selected from the group consisting of a vinyl, an acryloyl, a thiol, an alkene, a thiolester, an isocyanate, an isothiocyanate, an alkyl halide, a sulfonyl halide, an epoxide, an imidoesters, a fluorophenyl ester, a carbonate, a carbodiimide, a disulfide, a aziridines and any combinations thereof. In some embodiments, said first modification is selected from a vinylsulfone, a maleimide, an acrylate, a methacrylate, an epoxide and any combinations thereof. For example, said first modification is a maleimide or a vinylsulfone.

In some embodiments, said second modification is selected from the group consisting of a thiol, an amine, an azide, a hydrazide, a diene, a hydrazine, a hydroxylamines and any combinations thereof.

In some embodiments, said first hydrogel forming polymer and/or said second hydrogel forming polymer is selected from the group consisting of a polysaccharide, a derivative thereof, and any combinations thereof.

In some embodiments, said first hydrogel forming polymer and/or said second hydrogel forming polymer is selected from the group consisting of a hyaluronic acid, a chitosan, a chondroitin sulfate, an alginate, a carboxymethylcellulose, a dextran, a derivative thereof, and any combinations thereof.

In some embodiments, said first hydrogel forming polymer and/or said second hydrogel forming polymer is selected from the group consisting of a dextran, a hyaluronic acid, a derivative thereof, and any combinations thereof.

In some embodiments, said hydrogel is hydrolysable without the involvement of degradative enzymes.

In some embodiments, at least one of said first hydrogel forming polymer and/or said second hydrogel forming polymer comprises a degradable linker.

In some embodiments, said degradable linker comprises a hydrolysable functional group.

In some embodiments, said hydrolysable functional group is selected from an ester group, an anhydride group, and an amide group.

In some embodiments, said ester group is selected from an oxyester group and a thiolester group.

In some embodiments, said first hydrogel forming polymer derivative has a first average degree of modification (a first DM) of less than about 40% and said second hydrogel forming polymer derivative has a second average degree of modification (a second DM) of less than about 40%.

In some embodiments, a ratio between said first DM and said second DM is from about 3:1 to about 1:3.

In some embodiments, a molar ratio between said first hydrogel forming polymer derivative and said second hydrogel forming polymer derivative in said composition is from about 3:1 to about 1:3.

In some embodiments, said first hydrogel forming polymer derivative is a dextran derivative modified with one or more vinylsulfone groups, a hyaluronic acid derivative modified with one or more vinylsulfone groups, or a combination thereof, said second hydrogel forming polymer derivative is a dextran derivative modified with one or more thiol groups, a hyaluronic acid derivative modified with one or more thiol groups, or a combination thereof.

In some embodiments, said first hydrogel forming polymer and said second hydrogel forming polymer has a weight averaged molecular weight from about 1 kDa to about 500 kDa.

In some embodiments, said composition is a powder.

In some embodiments, said composition is a liquid composition, and a concentration of said first hydrogel forming polymer and/or said second hydrogel forming polymer in said liquid composition is from about 1% w/v to about 50% w/v.

In another aspect, the present disclosure provides a hydrogel for sustained release of a target agent, wherein said hydrogel is formed with the composition.

In some embodiments, said hydrogel further comprises the target agent.

In some embodiments, said target agent comprises a macromolecule.

In some embodiments, said target agent comprises a macromolecule of at least 80 kDa in molecular weight.

In some embodiments, said target agent comprises a protein or a polypeptide.

In some embodiments, at least about 20% of said target agent is free target agent not conjugated to the hydrogel.

In some embodiments, about less than 50% of said target agent is cumulatively released within an initial 24 hours from said hydrogel, and the remaining portion of said target agent is cumulatively released from said hydrogel in about 1 to about 36 months.

In some embodiments, the hydrogel comprises macroscopic hydrogel and micronized hydrogel.

In some embodiments, the hydrogel further comprises the micronized hydrogel. For example, the hydrogel further comprise the micronized hydrogel in a macroscopic hydrogel.

In another aspect, the present disclosure provides a method for producing a hydrogel, comprising: a) providing a composition, b) mixing said composition with a buffer to form a polymer solution; and c) subjecting said polymer solution to a condition enabling formation of the hydrogel.

In some embodiments, said subjecting comprises injecting said polymer solution in a subject in need thereof.

In some embodiments, said subjecting comprises incubating said composition at about 1° C. to about 45° C.

In some embodiments, said polymer solution further comprises said target agent. In another aspect, the present disclosure provides a method for producing a composition, comprising: a) crosslinking a precursor polymer with the degradable linker to obtain the first hydrogel forming polymer and/or first hydrogel forming polymer; and b) mixing said first hydrogel forming polymer and/or said second hydrogel forming polymer with an additional polymer, wherein said additional polymer is capable of reacting with said first hydrogel forming polymer and/or said second hydrogel forming polymer under a condition enabling formation of the hydrogel.

In another aspect, the present disclosure provides a method for sustained release of a target agent, comprising mixing said target agent with a composition to obtain a mixture, and subjecting said mixture to a condition enabling formation of a hydrogel capable of sustained release of said target agent.

In another aspect, the present disclosure provides a method for sustained release of a target agent, comprising enclosing said target agent in a hydrogel.

In another aspect, the present disclosure provides a kit, comprising: a) a composition; and b) a target agent to be sustained released by a hydrogel formed with the composition of a).

In another aspect, the present disclosure provides use of a composition for making a hydrogel.

In another aspect, the present disclosure provides use of a composition, or a hydrogel for sustained release of a target agent.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIG. 1 illustrates synthesis schemes of vinyl sulfone grafted dextran (DX-VS); and thiol grafted dextran (DX-DTT and PDT).

FIG. 2 illustrates synthesis schemes of modified functionalized dextran with an ester linkage (DX-O-SH and DX-O(Me)-SH).

FIG. 3 illustrates synthesis schemes of modified functionalized dextran with a degradable linker (DX-SH-VA-SH and DX-SH-VMA-SH).

FIG. 4 illustrates three forms of hydrogels.

FIG. 5 illustrates the swelling ratio (Wₜ/W₀) profiles of selected hydrolytically degradable hydrogel formulations varied in ester linker.

FIGS. 6A-6B illustrate the non-reducing SDS-PAGE showing the size of F-IgG (FITC-IgG, i.e., IgG labeled with fluorescein FITC) released from hydrolysable hydrogels under brightfield (A) and UV (B).

FIG. 7 illustrates the non-reducing SDS-PAGE showing the molecular weight of bevacizumab released from hydrolysable hydrogels.

FIG. 8 illustrates cumulative fractional release of IgG from non-degradable dextran based hydrogel formulations varied in initial polymer concentrations.

FIGS. 9A-9C illustrate effects of hydrogel degradation rate on the cumulative release profile of F-IgG, wherein, (A) change in swelling ratio due to bulk erosion. (B) cumulative release of F-IgG. (C) F-IgG release and hydrogel swelling of formulation 1 (C-1) and formulation 2 (C-2).

FIG. 10 illustrates cumulative release of F-IgG (A) and corresponding hydrogel swelling (B).

FIG. 11A illustrates in vivo pharmacokinetics protein bevacizumab and bevacizumab-encapsulated hydrogels; FIG. 11B illustrates in vitro release of bevacizumab from hydrogel.

FIG. 12 illustrates in vivo biocompatibility of protein-encapsulating hydrogels in rabbit eyes.

FIG. 13 illustrates schematics of showing the protein release from the hydrogel without crosslink degradation and during crosslink degradation.

FIG. 14 illustrates schematics of micronized hydrogel in macroscopic hydrogel.

FIG. 15 illustrates a format of the degradable linker.

FIG. 16 illustrates the NMR result of HA-MI.

FIG. 17 illustrates the swelling ratio of the hydrogel formed by HA-MI with different DMs;

FIG. 18 illustrates cumulative release of the hydrogel formed by HA-MI.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definition

The term “polymer”, as used herein, generally refers to a chemical compound or mixture of compounds formed by polymerization and consisting essentially of repeating structural units.

The term “hydrogel”, as used herein, generally refers to a gel or gel-like structure comprising one or more polymers suspended in an aqueous solution (e.g., water). All hydrogels possess some level of physical attraction between macromers as a result of hydrogen bonding and entanglements amongst one another. Usually a hydrogel intended for biomedical applications may be strengthened through additional electrostatic interactions or chemical cross-linking.

The term “sustained release”, as used herein, generally refers to a process for releasing a target agent relatively slowly over an extended period of time (e.g., in days, weeks, or months).

The term “degradable”, as used herein, generally refers to a property of a polymer structure (e.g., a polymer chain) of capable to be degraded under physiological conditions (e.g about 37° C. and pH is about 6.5˜8). The degradation may be chemical degradation (e.g hydrolytic cleavage), physical degradation (e.g., photon cleavage) or biological degradation (e.g. enzymatic cleavage). In some cases, the degradation may be hydrolysis, in some cases, the hydrolysis may happen at the crosslinks.

The term “hydrolysable” hydrogel, as used herein, generally refers to a polymer structure (e.g., a polymer chain) that can be at least partially hydrolyzed. For example, the hydrolysable structure may be formed by crosslinking linear, or branched non-hydrolysable precursor polymers using hydrolysable groups and/or crosslinkers comprising esters. The linear, or branched precursor polymers may be modified with one or more modifications. For instance, the hydrolysable functional group may be selected from an ester group, an anhydride group, and an amide group. For instance, the hydrolysable structure may be distinct from those polymers which the links between monomers are hydrolysable, such as Polylactic Acid (PLA) or poly (lactic-co-glycolic acid) (PLGA).

The term “hydrogel forming polymer”, as used herein, generally refers to a naturally occurring polymer or a synthetic polymer capable of forming a hydrogel. The hydrogel forming polymer can be classified according to their synthetic origins, composition, electrostatic nature and gel forming mechanism. In some cases, non-degradable hydrogel-forming polymers may have degradable regions built into their structure to impart finely controlled degradability. The hydrogel forming polymer may comprise at least a first hydrogel forming polymer and at least a second hydrogel forming polymer, and the first hydrogel forming polymer may be different from the second hydrogel forming polymer. The first hydrogel forming polymer may act with the second hydrogel forming polymer to form a hydrogel.

The term “hydrolysable”, as used herein, generally refers to a property of capable to be hydrolyzed. For example, a property of capable to be hydrolyzed at physiological temperature (30° C. to 40° C.) and pH (6.5 to 7.5) without catalyst, e.g., enzymes. Usually, hydrolysis is a chemical process in which a molecule of water breaks down one or more chemical bonds.

The term “electrophilic”, as used herein, generally refers to having an affinity for electron pairs. An electrophilic substance (e.g., molecule or portion of a molecule) may be an electron pair acceptor. In some embodiments, an electrophilic molecule or group may be selected from the group consisting of a vinyl, an acryloyl, a thiol, an alkene, a thiolester, an isocyanate, an isothiocyanate, an alkyl halide, a sulfonyl halide, an epoxide, an imidoesters, a fluorophenyl ester, a carbonate, a carbodiimide, a disulfide, a aziridines and any combinations thereof. In some embodiments, an electrophilic molecule or group may comprise a vinylsulfone, a maleimide, an acrylate, a methacrylate, an epoxide and any combinations thereof.

The term “nucleophilic”, as used herein, generally refers to having a property of capable of donating an electron pair to form a chemical bond in relation to a reaction with electrophilic substances. In some embodiments, the term may refer to a substance's nucleophilic character and an affinity for electriphiles. In some embodiments, a nucleophilic substance (e.g., molecule or portion of a molecule) may be selected from the group consisting of a thiol, an amine, an azide, a hydrazide, an amine, a diene, a hydrazine, a hydroxylamines and any combinations thereof. A nucleophilic molecule or group can act

The term “hydrophilic”, as used herein, generally refers to having an affinity for water, able to absorb or be wetted by water. A hydrophilic molecule or portion of a molecule is one whose interactions with water and other polar substances are more thermodynamically favorable than their interactions with oil or other hydrophobic solvents.

The term “ester group”, as used herein, generally refers to a chemical group derived from an acid (organic or inorganic) in which at least one —OH (hydroxyl) group is replaced by an —O-alkyl (alkoxy) group. For example, the ester group may be selected from an oxyester group and a thiolester group.

The term “average degree of modification (DM)”, as used herein, generally refers to the number of pendant groups per 100 repeating unit in a polymer. DM may reflect the degrees of modification of hydrogel forming polymer derivative.

The term “polydispersity”, as used herein, generally refers to a characteristic of polymers in term of disperse, or non-uniform, if the chain length of the polymer varies over a wide range of molecular masses. The polydispersity index (Ðx) may be calculated according to degree of polymerization. Ðx=Mw/Mn, where Mw is the weight average degree of polymerization and Mn is number average molecular weight. For example, the hydrogel forming polymer comprising the degradable backbone has a polydispersity of 4 or less.

The term “crosslink”, as used herein, generally refers to a bond that links one polymer chain to another. They can be covalent bonds or ionic bonds. “Polymer chains” may refer to synthetic polymers or natural polymers (such as hyaluronic acid). In polymer chemistry, when a polymer is said to be “cross-linked”, it usually means that the entire bulk of the polymer has been exposed to the cross-linking method.

The term “precursor polymer”, as used herein, generally refers to a polymer used to form another polymer structure or to be further modified. This material is capable of further polymerization by reactive groups to form structures of higher molecular weight.

The term “composition”, as used herein, generally refers to a product (liquid or solid-state) of various elements or ingredients.

The term “biocompatible” or “biocompatibility”, as used herein, generally refers to a condition of being compatible with a living tissue or a living system by not being toxic, injurious, or physiologically reactive and/or not causing immunological rejection.

The term “about”, when used in the context of numerical values, generally refers to a value less than 1% to 15% (e.g., less than 1%, less than 2%, less than 3%, less than 4%, less than 5%, less than 6%, less than 7%, less than 8%, less than 9%, less than 10%, less than 11%, less than 12%, less than 13%, less than 14%, or less than 15%) above or below an indicated value.

Where a range of values (e.g., a numerical range) is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

As used herein, the singular forms “a,” “and,” and “the” include plural referents unless the context clearly dictates otherwise. Thus, for example, reference to “a particle” includes a plurality of such particles and reference to “the sequence” includes reference to one or more said sequences and equivalents thereof known to those skilled in the art, and so forth.

As will be understood by those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

The present disclosure provides compositions comprising one or more hydrogel forming polymers and methods for making and using the same. And the present disclosure provides a hydrogel and methods for making and using the same.

In one aspect, the present disclosure provides a composition which may comprise at least a (e.g., one, two, three, four, five, six, seven, eight, night, ten or more) first hydrogel forming polymer and at least a (e.g., one, two, three, four, five, six, seven, eight, night, ten or more) second hydrogel forming polymer, said first hydrogel forming polymer is capable of reacting with said hydrogel forming second polymer to form said hydrogel, and said hydrogel is degradable (e.g., hydrolysable, enzymatically degradable, or otherwise cleavable.) and enables sustained release of a target agent.

In the present disclosure, the first hydrogel forming polymer may comprise a first hydrogel forming polymer derivative, said first hydrogel forming polymer derivative may comprise a first modification, and the first hydrogel forming polymer derivative may be electrophilic.

In some embodiments, the first modification may be selected from the group consisting of a vinyl, an acryloyl, a thiol, an alkene, a thiolester, an isocyanate, an isothiocyanate, an alkyl halide, a sulfonyl halide, an epoxide, an imidoesters, a fluorophenyl ester, a carbonate, a carbodiimide, a disulfide, a aziridines and any combinations thereof. In some embodiments, the first modification may be selected from the group consisting of a vinyl, a thiol, an alkene, a thiolester, an isocyanate, an isothiocyanate, an alkyl halide, a sulfonyl halide, an epoxide, an imidoesters, a fluorophenyl ester, a carbonate, a carbodiimide, a disulfide, a aziridines and any combinations thereof.

In some embodiments, said first modification is selected from a vinylsulfone, a maleimide, an acrylate, a methacrylate, an epoxide and any combinations thereof. In some embodiments, said first modification is selected from a maleimide, an acrylate, a methacrylate, an epoxide and any combinations thereof. For example, said first modification is a maleimide or a vinylsulfone.

In some embodiments, the first modification may be selected from the group consisting of a vinyl, a maleimide, an acrylate, a methacrylate, an epoxide, a thiol, an alkene, a thiolester, an isocyanate, an isothiocyanate, an alkyl halide, a sulfonyl halide, an epoxide, an imidoesters, a fluorophenyl ester, a carbonate, a carbodiimide, a disulfide, a aziridines and any combinations thereof. For example, said first modification is a maleimide or a vinylsulfone.

In the present disclosure, the second hydrogel forming polymer may comprise a second hydrogel forming polymer derivative, said second hydrogel forming polymer derivative may comprise a second modification, and the second hydrogel forming polymer derivative may be nucleophilic.

In some embodiments, the second modification may be selected from the group consisting of a thiol, an amine, an azide, a hydrazide, an amine, a diene, a hydrazine, a hydroxylamines and any combinations thereof. In some embodiments, the second modification may be selected from the group consisting of an amine, an azide, a hydrazide, an amine, a diene, a hydrazine, a hydroxylamines and any combinations thereof.

In some embodiments, the first modification may be selected from the group consisting of said first modification is selected from the group consisting of a vinyl, an acryloyl (e.g., a maleimide, an acrylate, a methacrylate, an epoxide and any combinations thereof), a thiol, an alkene, a thiolester, an isocyanate, an isothiocyanate, an alkyl halide, a sulfonyl halide, an epoxide, an imidoesters, a fluorophenyl ester, a carbonate, a carbodiimide, a disulfide, a aziridines and any combinations thereof and the second modification may be selected from the group consisting of a thiol, an amine, an azide, a hydrazide, an amine, a diene, a hydrazine, a hydroxylamines and any combinations thereof.

In some embodiments, the first modification may be selected from the group consisting of a vinyl, an acryloyl (e.g., a vinylsulfone, a maleimide, an acrylate, a methacrylate, an epoxide and any combinations thereof), a thiol, an alkene, a thiolester, an isocyanate, an isothiocyanate, an alkyl halide, a sulfonyl halide, an epoxide, an imidoesters, a fluorophenyl ester, a carbonate, a carbodiimide, a disulfide, a aziridines and any combinations thereof and the second modification may be selected from the group consisting of an amine, an azide, a hydrazide, an amine, a diene, a hydrazine, a hydroxylamines and any combinations thereof.

In some embodiment, in the composition, the first modification may comprise one or more vinylsulfone and the second modification may comprise one or more thiols.

In some embodiments, the first polymer derivative may be capable of reacting with the second polymer derivative to form the hydrogel.

In the present disclosure, a mass ratio between the first hydrogel forming polymer and the second hydrogel forming polymer in the composition may be less than about 1 (e.g., less than about 0.95, less than about 0.9, less than about 0.85, less than about 0.8, less than about 0.75, less than about 0.7, less than about 0.65, less than about 0.6, less than about 0.55, less than about 0.5, less than about 0.45, less than about 0.4, less than about 0.35, less than about 0.3, less than about 0.25, less than about 0.2, less than about 0.15, less than about 0.1, less than about 0.05, or less).

In some embodiments, the mass ratio between the first hydrogel forming polymer and the second hydrogel forming polymer in the composition may be from about 0 to about 1, e.g., from about 0 to about 0.99, from about 0 to about 0.95, from about 0 to about 0.9, from about 0 to about 0.8, from about 0 to about 0.7, from about 0 to about 0.6, from about 0 to about 0.5, from about 0 to about 0.49, from about 0 to about 0.45, from about 0 to about 0.4, from about 0 to about 0.3, from about 0 to about 0.2, from about 0 to about 0.1, from about 0.1 to about 1, from about 0.2 to about 1, from about 0.3 to about 1, from about 0.4 to about 1, from about 0.5 to about 1, from about 0.51 to about 1, from about 0.55 to about 1, from about 0.6 to about 1, from about 0.7 to about 1, from about 0.8 to about 1, from about 0.9 to about 1, from about 0.1 to about 0.5, from about 0.1 to about 0.49, from about 0.1 to about 0.45, from about 0.1 to about 0.4, from about 0.2 to about 0.3, from about 0.5 to about 0.99, from about 0.51 to about 0.99, from about 0.6 to about 0.9, or from about 0.7 to about 0.8, etc.

In some embodiments, the mass ratio between the first hydrogel forming polymer and the second hydrogel forming polymer in the composition may be about 0.95, about 0.9, about 0.85, about 0.8, about 0.75, about 0.7, about 0.67, about 0.65, about 0.6, about 0.55, about 0.5, about 0.45, about 0.4, about 0.35, about 0.3, about 0.25, about 0.2, about 0.15, about 0.1, or about 0.05, etc.

In the present disclosure, the first hydrogel forming polymer derivative may be capable of reacting with the second hydrogel forming polymer derivative to form the hydrogel.

In the present disclosure, the first hydrogel forming polymer may be selected from the group consisting of a polysaccharide, a derivative thereof, and any combinations thereof.

In the present disclosure, the second hydrogel forming polymer may be selected from the group consisting of a polysaccharide, a derivative thereof, and any combinations thereof.

In some cases, the polysaccharide may be homoglycans, i.e. polysaccharides having a main chain consisting of one single sugar, e.g. colominic acid; or, may be heteroglycans, i.e. polysaccharides having more than one sugar residue in the main chain in either alternating or less regular sequence; e.g. Gellans; Succinoglycans; Arabinogalactans; Tragacanth or gum tragacanth or traganth from Astragalus; Gum Karaya from Sterculia urens; Gum Ghatti from Anogeissus latifolia and the derivatives thereof.

In the present disclosure, the first hydrogel forming polymer may be selected from the group consisting of a hyaluronic acid, a chitosan, a chondroitin sulfate, an alginate, a carboxymethylcellulose, a dextran, a derivative thereof, and any combinations thereof. In some cases, the first hydrogel forming polymer may be selected from the group consisting of a hyaluronic acid, a chitosan, a chondroitin sulfate, an alginate, a carboxymethylcellulose, a dextran, a derivative thereof, and any combinations thereof.

In some cases, the first hydrogel forming polymer in the composition may be selected from the group consisting of a dextran, a hyaluronic acid, a derivative thereof, and any combinations thereof. In some cases, the first hydrogel forming polymer may be selected from the group consisting of a hyaluronic acid, a derivative thereof, and any combinations thereof. In some cases, the first hydrogel forming polymer may be a hyaluronic acid.

In the present disclosure, the second hydrogel forming polymer may be selected from the group consisting of a dextran, a hyaluronic acid, a chitosan, a chondroitin sulfate, an alginate, a carboxymethylcellulose, a dextran, a derivative thereof, and any combinations thereof. In some cases, the second hydrogel forming polymer may be selected from the group consisting of a hyaluronic acid, a chitosan, a chondroitin sulfate, an alginate, a carboxymethylcellulose, a dextran, a derivative thereof, and any combinations thereof.

In some cases, the second hydrogel forming polymer in the composition may be selected from the group consisting of a dextran, a hyaluronic acid, a derivative thereof, and any combinations thereof. In some cases, the second hydrogel forming polymer may be selected from the group consisting of a hyaluronic acid, a dextran, a derivative thereof, and any combinations thereof. In some cases, the first hydrogel forming polymer may be a hyaluronic acid.

In the present disclosure, the first hydrogel forming polymer in the composition may be selected from the group consisting of a dextran, a hyaluronic acid, a derivative thereof, and any combinations thereof and the second hydrogel forming polymer in the composition may be selected from the group consisting of a dextran, a hyaluronic acid, a derivative thereof, and any combinations thereof.

In some cases, the first hydrogel forming polymer may be selected from the group consisting of a hyaluronic acid, a derivative thereof and the second hydrogel forming polymer in the composition may be selected from the group consisting of a dextran, a hyaluronic acid, a derivative thereof, and any combinations thereof. In some cases, the first hydrogel forming polymer may be selected from the group consisting of a dextran, a hyaluronic acid, a derivative thereof and the second hydrogel forming polymer in the composition may be selected from the group consisting of a hyaluronic acid, a derivative thereof, and any combinations thereof.

In the present disclosure, the first hydrogel forming polymer derivative may have an first average degree of modification (a first DM) of less than about 40% (e.g. less than about 40%, less than about 35%, less than about 30%, less than about 25%, less than about 20%, less than about 19%, less than about 18%, less than about 17%, less than about 16%, less than about 15%, less than about 14%, less than about 13%, less than about 12%, less than about 11%, less than about 10%, less than about 8%, less than about 6%, less than about 5%, less than about 4%, less than about 2%, less than about 0.5% or less).

In some cases, the first hydrogel forming polymer derivative may have an average DM from about 0% to about 40% (e.g., from about 0.001% to about 19.5%, from about 0.001% to about 4.9%, from about 0.5% to about 5%, from about 5.5% to about 19.5%, from about 8% to about 19%, from about 9% to about 20%, from about 8.5% to about 18%, or, from about 8.5% to about 17.5%, from about 0.001% to about 39.5%, from about 0.001% to about 35%, from about 0.001% to about 30%, from about 0.001% to about 7.5%, from about 9.5% to about 20%, from about 20% to about 30%, or, from about 20% to about 40%, from about 10% to about 40%, etc.).

In the present disclosure, the second hydrogel forming polymer derivative may have an second average degree of modification (a second DM) of less than about 40% (e.g. less than about 40%, less than about 35%, less than about 30%, less than about 25%, less than about 20%, less than about 19%, less than about 18%, less than about 17%, less than about 16%, less than about 15%, less than about 14%, less than about 13%, less than about 12%, less than about 11%, less than about 10%, less than about 8%, less than about 6%, less than about 5%, less than about 4%, less than about 2%, less than about 0.5% or less).

In some cases, the second hydrogel forming polymer derivative may have an average DM from about 0% to about 40% (e.g., from about 0.001% to about 1⁹0.5%, from about 0.001% to about 4.5%, from about 0.001% to about 4.9%, from about 0.5% to about 5%, from about 5% to about 8%, from about 5.1% to about 7.9%, from about 5.5% to about 19.9%, from about 8% to about 19.9%, from about 8.1% to about 19.9%, from about 8.5% to about 18%, or, from about 8.5% to about 17.5%, from about 20% to about 25%, from about 20% to about 30%, from about 20% to about 35%, from about 20% to about 40%, from about 10% to about 40%, etc.).

In the present disclosure, a ratio between the first DM and the second DM may be from about 3:1 to about 1:3(e.g. from about 3:1 to about 1:3, from about 3:1.5 to about 1:3, from about 3:2 to about 1:3, from about 3:2.5 to about 1:3, from about 3:1 to about 1:2.5, from about 3:1 to about 1:2, from about 3:1 to about 1:1.5, from about 2.5:1 to about 1:3, from about 2:1 to about 1:3, from about 1.5:1 to about 1:3 etc.).

In the present disclosure, a molar ratio between the first hydrogel forming polymer derivative and the second hydrogel forming polymer derivative in the composition may be from about 3:1 to about 1:3(e.g. from about 3:1 to about 1:3, from about 3:1.5 to about 1:3, from about 3:2 to about 1:3, from about 3:2.5 to about 1:3, from about 3:1 to about 1:2.5, from about 3:1 to about 1:2, from about 3:1 to about 1:1.5, from about 2.5:1 to about 1:3, from about 2:1 to about 1:3, from about 1.5:1 to about 1:3 etc.).

In the present disclosure, a volume ratio between the first hydrogel forming polymer derivative and the second hydrogel forming polymer derivative in the composition may be from about 10:1 to about 1:10 (e.g. from about 10:1 to about 1:10, from about 8:1 to about 1:10, from about 6:1 to about 1:10, from about 5:1 to about 1:10, from about 4:1 to about 1:10, from about 3:1 to about 1:10, from about 2:1 to about 1:10, from about 1.75:1 to about 1:10, from about 1.5:1 to about 1:10, from about 1.25:1 to about 1:10, from about 1:1 to about 1:10, from about 1:1.25 to about 1:10, from about 1:1.5 to about 1:10, from about 1:1.75 to about 1:10, from about 1:2 to about 1:10, from about 1:3 to about 1:10, from about 1:4 to about 1:10, from about 1:5 to about 1:10, from about 6:1 to about 1:6, from about 5:1 to about 1:5, from about 4:1 to about 1:4, from about 3:1 to about 1:3, from about 2:1 to about 1:2, from about 1.75:1 to about 1:1.75, from about 1.5:1 to about 1:1.5, from about 1.25:1 to about 1:1.25, or from about 1.1:1 to about 1:1.1, etc.).

In some cases, the first hydrogel forming polymer derivative may be modified with one or more vinylsulfone groups and the second hydrogel forming polymer derivative may be modified with one or more thiol groups. In some cases, the first hydrogel forming polymer derivative may be modified with one or more maleimide groups and the second hydrogel forming polymer derivative may be modified with one or more thiol groups. In some cases, the first hydrogel forming polymer derivative may be modified with one or more acrylate groups and the second hydrogel forming polymer derivative may be modified with one or more amine groups. In some cases, the first hydrogel forming polymer derivative may be modified with one or more methacrylate groups and the second hydrogel forming polymer derivative may be modified with one or more amine groups.

In the present disclosure, the first hydrogel forming polymer derivative may be a dextran derivative modified with one or more vinylsulfone groups, a hyaluronic acid derivative modified with one or more vinylsulfone groups, a dextran derivative modified with one or more maleimide groups, a hyaluronic acid derivative modified with one or more maleimide groups, a dextran derivative modified with one or more acrylate groups, a hyaluronic acid derivative modified with one or more acrylate groups, a dextran derivative modified with one or more methacrylate groups, a hyaluronic acid derivative modified with one or more methacrylate groups, or a combination thereof.

In the present disclosure, the second hydrogel forming polymer derivative may be a dextran derivative modified with one or more thiol groups, a hyaluronic acid derivative modified with one or more thiol groups, a dextran derivative modified with one or more amine groups, a hyaluronic acid derivative modified with one or more amine groups, or a combination thereof.

In the present disclosure, the first hydrogel forming polymer derivative may be a dextran derivative modified with one or more vinylsulfone groups, a hyaluronic acid derivative modified with one or more vinylsulfone groups, a dextran derivative modified with one or more maleimide groups, a hyaluronic acid derivative modified with one or more maleimide groups, a dextran derivative modified with one or more acrylate groups, a hyaluronic acid derivative modified with one or more acrylate groups, a dextran derivative modified with one or more methacrylate groups, a hyaluronic acid derivative modified with one or more methacrylate groups, or a combination thereof, and the second hydrogel forming polymer derivative may be a dextran derivative modified with one or more thiol groups, a hyaluronic acid derivative modified with one or more thiol groups, a dextran derivative modified with one or more amine groups, a hyaluronic acid derivative modified with one or more amine groups, or a combination thereof.

In the present disclosure, the first hydrogel forming polymer derivative may be a dextran derivative modified with one or more vinylsulfone groups, a hyaluronic acid derivative modified with one or more vinylsulfone groups, a dextran derivative modified with one or more maleimide groups, a hyaluronic acid derivative modified with one or more maleimide groups, a dextran derivative modified with one or more acrylate groups, a hyaluronic acid derivative modified with one or more acrylate groups, a dextran derivative modified with one or more methacrylate groups, a hyaluronic acid derivative modified with one or more methacrylate groups, or a combination thereof, and the second hydrogel forming polymer derivative may be a hyaluronic acid derivative modified with one or more thiol groups, a dextran derivative modified with one or more thiol groups, a dextran derivative modified with one or more amine groups, a hyaluronic acid derivative modified with one or more amine groups, or a combination thereof.

In the present disclosure, the first hydrogel forming polymer derivative may be a hyaluronic acid derivative modified with one or more vinylsulfone groups, a dextran derivative modified with one or more maleimide groups, a hyaluronic acid derivative modified with one or more maleimide groups, a dextran derivative modified with one or more acrylate groups, a hyaluronic acid derivative modified with one or more acrylate groups, a dextran derivative modified with one or more methacrylate groups, a hyaluronic acid derivative modified with one or more methacrylate groups, or a combination thereof, and the second hydrogel forming polymer derivative may be a dextran derivative modified with one or more thiol groups, a hyaluronic acid derivative modified with one or more thiol groups, a dextran derivative modified with one or more amine groups, a hyaluronic acid derivative modified with one or more amine groups, or a combination thereof.

For example, the first hydrogel forming polymer derivative may be a hyaluronic acid derivative modified with one or more maleimide groups, and the second hydrogel forming polymer derivative may be a dextran derivative modified with one or more thiol groups.

For example, the first hydrogel forming polymer derivative may be a dextran derivative modified with one or more maleimide groups, and the second hydrogel forming polymer derivative may be a hyaluronic acid derivative modified with one or more thiol groups.

In the present disclosure, said hydrogel is hydrolysable without the involvement of degradative enzymes.

In the present disclosure, the at least one of said first hydrogel forming polymer and/or said second hydrogel forming polymer comprises a degradable linker. In some embodiments, the degradable linker may be hydrolysable. In another embodiments, the hydrolysis may happen at the crosslinks.

In the present disclosure, the degradable linker may comprise a hydrolysable functional group. For example, the hydrolysable functional group may be selected from an ester group, an anhydride group, and an amide group.

In the present disclosure, the ester group may be selected from an oxyester group and a thiolester group. For example, the oxyester group may have a functional group of —COOR, and the thiolester group may have a functional group of R—S—CO—R′, which may be the product of esterification between a carboxylic acid and a thiol.

In the present disclosure, the first hydrogel forming polymer may have a weight averaged molecular weight from about 1 kDa to about 500 kDa (e.g. from about 1 kDa to about 500 kDa, from about 3 kDa to about 500 kDa, from about 5 kDa to about 500 kDa, from about 7 kDa to about 500 kDa, from about 10 kDa to about 500 kDa, from about 50 kDa to about 500 kDa, from about 100 kDa to about 500 kDa, from about 150 kDa to about 500 kDa, from about 200 kDa to about 500 kDa, from about 250 kDa to about 500 kDa, from about 300 kDa to about 500 kDa, from about 350 kDa to about 500 kDa, from about 400 kDa to about 500 kDa, from about 450 kDa to about 500 kDa, from about 1 kDa to about 39 kDa, from about 41 kDa to about 200 kDa, or from about 41 kDa to about 500 kDa).

In some case, the first hydrogel forming polymer may have a weight averaged molecular weight less than 500 kDa (e.g., less than 490 kDa, less than 480 kDa, less than 450 kDa, less than 400 kDa, less than 300 kDa, less than 200 kDa, less than 150 kDa, less than 100 kDa, less than 50 kDa, less than 40 kDa, less than 30 kDa, less than 20 kDa, less than 10 kDa, or less). In some case, the first hydrogel forming polymer may have a weight averaged molecular weight more than 1 kDa (e.g., more than 1 kDa, more than 5 kDa, more than 10 kDa, more than 20 kDa, more than 30 kDa, more than 40 kDa, more than 41 kDa, more than 45 kDa, more than 50 kDa, more than 100 kDa, more than 200 kDa, more than 300 kDa, more than 400 kDa, or more).

In the present disclosure, the second hydrogel forming polymer may have a weight averaged molecular weight from about 1 kDa to about 500 kDa (e.g. from about 1 kDa to about 500 kDa, from about 3 kDa to about 500 kDa, from about 5 kDa to about 500 kDa, from about 7 kDa to about 500 kDa, from about 10 kDa to about 500 kDa, from about 50 kDa to about 500 kDa, from about 100 kDa to about 500 kDa, from about 150 kDa to about 500 kDa, from about 200 kDa to about 500 kDa, from about 250 kDa to about 500 kDa, from about 300 kDa to about 500 kDa, from about 350 kDa to about 500 kDa, from about 400 kDa to about 500 kDa, from about 450 kDa to about 500 kDa, from about 1 kDa to about 39 kDa, from about 41 kDa to about 200 kDa, or from about 41 kDa to about 500 kDa).

In some case, the second hydrogel forming polymer may have a weight averaged molecular weight less than 500 kDa (e.g., less than 490 kDa, less than 480 kDa, less than 450 kDa, less than 400 kDa, less than 300 kDa, less than 200 kDa, less than 150 kDa, less than 100 kDa, less than 50 kDa, less than 40 kDa, less than 30 kDa, less than 20 kDa, less than 10 kDa, or less). In some case, the first hydrogel forming polymer may have a weight averaged molecular weight more than 1 kDa (e.g., more than 1 kDa, more than 5 kDa, more than 10 kDa, more than 20 kDa, more than 30 kDa, more than 40 kDa, more than 41 kDa, more than 45 kDa, more than 50 kDa, more than 100 kDa, more than 200 kDa, more than 300 kDa, more than 400 kDa, or more).

In some cases, the composition may be a powder.

In some cases, the composition may be a liquid composition, and a concentration of the one or more hydrogel forming polymers in the liquid composition is from about 1% w/v to about 30% w/v (e.g. from about 1% w/v to about 50% w/v, from about 5% w/v to about 50% w/v, from about 10% w/v to about 50% w/v., from about 15% w/v to about 50% w/v., from about 20% w/v to about 50% w/v, from about 25% w/v to about 50% w/v., from about 30% w/v to about 50% w/v., from about 35% w/v to about 50% w/v., from about 40% w/v to about 50% w/v, from about 45% w/v to about 50% w/v, from about 1% w/v to about 45% w/v, from about 1% w/v to about 40% w/v, from about 1% w/v to about 35% w/v, from about 1% w/v to about 30% w/v, from about 1% w/v to about 25% w/v, from about 1% w/v to about 20% w/v, from about 11% w/v to about 15% w/v, from aboutl % w/v to about 10% w/v, from about 1% w/v to about 5% w/v, etc).

In the present disclosure, the hydrogel forming polymer comprising the degradable backbone may be formed by grafting the precursor polymers with the degradable linker, and the degradable linker may enable formation of degradable linkage between the precursor polymers.

In some cases, the precursor polymer may be hydrophilic and/or water soluble.

In some cases, the precursor polymer may be non-hydrolysable, enzymatically non-degradable, or otherwise non-cleavable. For example, when the degradable linker was degraded by hydrolyze, enzyme and other clear pathways, the precursor polymer may not be affected and may maintain the structure of the degradable backbone.

In the present disclosure, the precursor polymer may be selected from the group consisting of a polysaccharide, a derivative thereof, and any combinations thereof.

In some cases, the precursor polymer may be selected from the group consisting of a dextran, a hyaluronic acid, a derivative thereof, and any combinations thereof.

In the present disclosure, the precursor polymer may be a derivative comprising one or more (e.g. one, two, three, four, five, six, seven, eight, nine, ten or more) modifications, and a degree of modification of the precursor polymer is less than about 40% (e.g. less than about 40%, less than about 35%, less than about 30%, less than about 25%, less than about 20%, less than about 18%, less than about 16%, less than about 14%, less than about 12%, less than about 10%, less than about 8%, less than about 6%, less than about 4%, less than about 2%, less than about 1% or less).

In the present disclosure, the modification of the precursor polymer may be selected from the group consisting of an acrylate, a methacrylate, a maleimide, a vinylsulfone, a thiol, an amine, and any combinations thereof.

In the present disclosure, the degradable linker may comprise two or more (e.g. two, three, four, five, six, seven, eight, nine, ten or more) modifications, and a degree of modification of the degradable linker is less than about 40% (e.g. less than about 40%, less than about 35%, less than about 30%, less than about 25%, less than about 20%, less than about 15%, less than about 10%, less than about 5%, less than about 1% or less).

In the present disclosure, the modification of the degradable linker may be selected from the group consisting of an acrylate, a methacrylate, a maleimide, a vinylsulfone, a thiol, an amine, and any combinations thereof.

In the present disclosure, the precursor polymer may be a dextran derivative modified with one or more vinylsulfone groups, a hyaluronic acid derivative modified with one or more vinylsulfone groups, derivative modified with one or more (e.g. one, two, three, four, five, six, seven, eight, nine, ten or more) vinylsulfone groups, or a combination thereof, and the degradable linker comprises two or more (e.g. two, three, four, five, six, seven, eight, nine, ten or more) thiol group modifications. For example, the vinylsulfone groups may have a functional group of

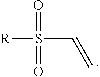

In some cases, the precursor polymer may be a hyaluronic acid derivative modified with one or more (e.g. one, two, three, four, five, six, seven, eight, nine, ten or more) thiol groups, a dextran derivative modified with one or more (e.g. one, two, three, four, five, six, seven, eight, nine, ten or more) thiol groups, or a combination thereof, and the degradable linker comprises two or more (e.g. two, three, four, five, six, seven, eight, nine, ten or more) vinylsulfone group modifications.

In the present disclosure, the degradable linker may be selected from a divinyl methacrylate, a divinyl acrylate, and a derivative thereof.

In some cases, the degradable linker may be selected form the following groups:

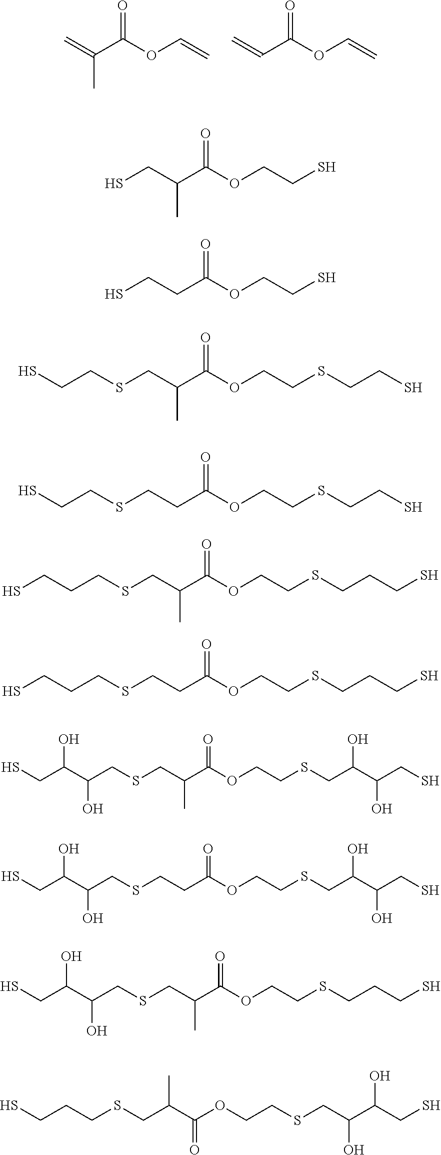

In the present disclosure, the degradable linker may comprise a modulator, an ester. In some cases, the degradable linker may further comprise a linker. In some cases, said ester may be modified with said modulator. For example, one side of said ester may be modified with said modulator, or, both two sides of said ester may be modifies with said modulator. In some cases, the degradable linker having said ester modified on both sides with said modulator may be significantly more stabilized than the degradable linker having said ester modified on one side with said modulator. In some cases, the degradable linker having said ester modified on both sides with said modulator may show a slower ester hydrolysis rate than the degradable linker having said ester modified on one side with said modulator.

In some cases, the degradable linker may comprise a modulator, an ester, and a linker. For example, the degradable linker may comprise the format shown in FIG. 15.

In some cases, the two modulators may be the same or be the different. In some cases, the two modulators may be the same.

In some cases, said ester may be selected form the following groups:

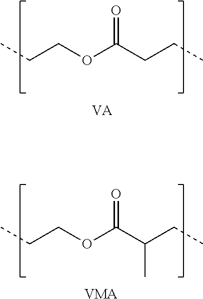

In some cases, said modulator may be hydrophobic or be hydrophilic. In some cases, the hydrophobic modulator may increase the stability of the degradable linker than the hydrophilic modulator. In some cases, the hydrophobic modulator may reduce the solubility of the degradable linker in the aqueous environment.

In some cases, said modulator may be electron withdrawing or electron donating.

In some cases, said modulator may be selected form the following groups:

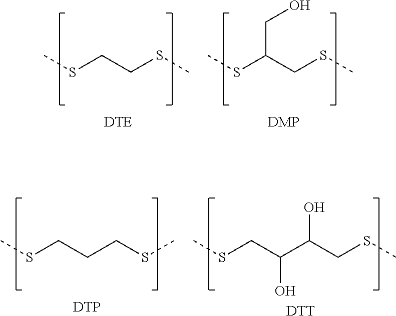

In some cases, said linker may be selected form the following groups:

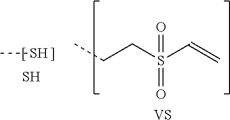

In some cases, the hydrogel forming polymer derivative may comprise a modification, where the modification is of formula (1), (2), (3), (4) or a combination of them

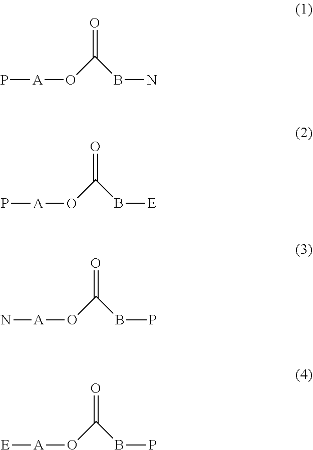

Wherein P is the polymer, A is the linker or the modifier or a combination of both, B is the linker or the modifier or a combination of both that is the same or different from A,

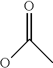

is the ester, N is the nucleophile, E is the electrophile.

In some cases, the concentration of the precursor polymer may have an influence on the hydrolytic degradation of the hydrogel forming polymer.

In some cases, the average degree of modification (DM) of the hydrogel forming polymer (e.g. the precursor polymer) may have an influence on the hydrolytic degradation of the hydrogel forming polymer.

In some cases, the average molecular weight (Mw) of the hydrogel forming polymer (e.g. the precursor polymer) may have an influence on the hydrolytic degradation of the hydrogel forming polymer.

In another aspect, the present disclosure provides a hydrogel for sustained release of a target agent, wherein the hydrogel may be formed with the composition.

In the present disclosure, the hydrogel may disassociate as the precursor polymer or the crosslinker is degraded. In some cases, the molecular weight of degradation products of the hydrogel may span over a wide range of values.

In the present disclosure, the release of proteins from hydrogel meshwork before and after crosslink degradation can be illustrated in FIG. 13, wherein, lines represent the polymer network, dotted lines represent the polymer after crosslink degradation, pale back ground represents the water, trigonal objects represent the protein and filled circle represent the crosslinks.

In the present disclosure, the hydrogel further may comprise the target agent.

In some cases, the target agent comprises a macromolecule of at least about 80 kDa in molecular weight, e.g., at least about 80 kDa in molecular weight, at least about 90 kDa in molecular weight, at least about 100 kDa in molecular weight, at least about 120 kDa in molecular weight, at least about 150 kDa in molecular weight, at least about 180 kDa in molecular weight, at least about 200 kDa in molecular weight, at least about 250 kDa in molecular weight, at least about 300 kDa, or more in molecular weight.

In some cases, the target agent comprises a macromolecule. For example, the target agent may comprise a protein or a polypeptide.

In the present disclosure, at least about 20% (e.g., at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 910%, at least about 92%, at least about 93%, at least about 94%, at least about 96%, at least about 98%, at least about 99%, or more) of said target agent may be free target agent (e.g., protein) not conjugated to said hydrogel. In some embodiments, at least about 80% (e.g., at least about 80%, at least about 85%, at least about 90%, at least about 91%, at least about 92%, at least about 93%, at least about 94%, at least about 96%, at least about 98%, at least about 99%, or more) of said target agent may be free target agent (e.g., protein) not conjugated to said hydrogel.

In the present disclosure, about less than 50% (e.g. less than about 50%, less than about 45%, less than about 40%, less than about 35%, less than about 30%, less than about 25%, less than about 20%, less than about 15%, less than about 10%, less than about 5%, less than about 1% or less) of the target agent may be cumulatively released within an initial 24 hours (e.g. within an initial 24 hours, 22 hours, 20 hours, 18 hours, 16 hours, 14 hours, 12 hours, 10 hours, 8 hours, 6 hours, 4 hours, 2 hours or less) from the hydrogel, and the remaining portion of the target agent may be cumulatively released from the hydrogel in about 1 to about 36 months (e.g. about 1 to about 36 months, about 1 to about 30 months, about 1 to about 24 months, about 1 to about 18 months, about 1 to about 12 months, about 1 to about 10 months, about 1 to about 9 months, about 1 to about 8 months, about 1 to about 7 months, about 1 to about 6 months, about 1 to about 5 months, about 1 to about 4 months, about 1 to about 2 months, about 1 to about 3 months, about 4 to about 36 months, about 5 to about 36 months, about 6 to about 36 months, about 7 to about 36 months, about 8 to about 36 months, about 9 to about 36 months, about 10 to about 36 months, about 12 to about 36 months, about 14 to about 36 months, about 16 to about 36 months, about 18 to about 36 months, about 18 to about 24 months, about 20 to about 24 months, about 22 to about 24 months).

In the present disclosure, the target agent may be cumulatively released from the hydrogel in more than 1 day, more than 1 week, more than 1 month, more than 3 months, more than 4 months, more than 5 months, more than 6 months, more than 7 months, more than 8 months, more than 9 months, more than 10 months, more than 11 months, more than 12 months, more than 24 months, or more than 36 months.

In the present disclosure, the initial 24 hours (e.g. within an initial 24 hours, 22 hours, 20 hours, 18 hours, 16 hours, 14 hours, 12 hours, 10 hours, 8 hours, 6 hours, 4 hours, 2 hours or less) may be started to timing once the hydrogel containing the target agent is formed.

In the present disclosure, the hydrogel may be a premade hydrogel, or a composition of polymers which upon mixing and injection will form a hydrogel in the body. In some cases, the hydrogel may be a hydrogel of micron size (micronized hydrogel), or a regular hydrogel of about centimeter or larger in size (macroscopic hydrogel). In other cases, the solvent of the hydrogel or the polymer may contain the micronized hydrogel (micronized hydrogel in a macroscopic hydrogel).

In some example, the solvent in the above-mentioned hydrogel microsphere can contain proteins, or contains a protein-encapsulating micronized hydrogel.

In some cases, the macroscopic hydrogel may be capable to entrap micronized hydrogel. In some cases, the micronized hydrogel may capable to physically entrap macromolecules.

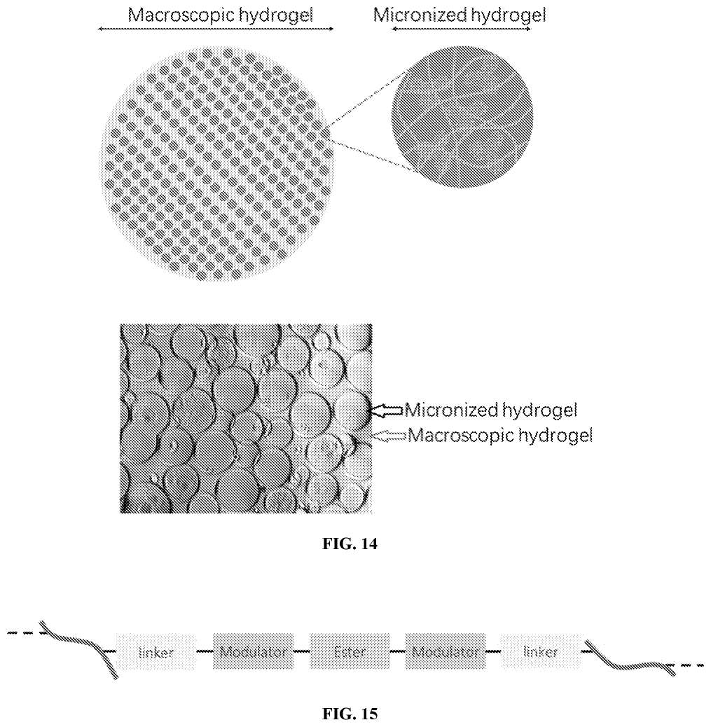

For example, the hydrogel may comprise an in-situ forming macroscopic hydrogel and a preformed micronized hydrogel (FIG. 14). The in-situ forming macroscopic hydrogel may entrap the preformed micronized hydrogel, and the preformed micronized hydrogel may physically entrap macromolecules.

In another aspect, the present disclosure provides a method for producing a hydrogel, and the method may comprise: a) providing the composition of the present disclosure; b) mixing the composition with a buffer to form a polymer solution; and c) subjecting the polymer solution to a condition enabling formation of the hydrogel.

In the present disclosure, the subjecting may comprise injecting the polymer solution in a subject in need thereof.

In some cases, the subjecting may comprise incubating the composition at about 1° C. to about 45° C. (e.g., about 1° C. to about 10° C., about 1° C. to about 8° C., about 1° C. to about 6° C., about 2° C. to about 6° C., about 3° C. to about 5° C., about 1° C. to about 45° C., about 2° C. to about 45° C., about 3° C. to about 45° C., about 4° C. to about 45° C., about 6° C. to about 45° C., about 8° C. to about 45° C., about 10° C. to about 45° C., about 15° C. to about 45° C., about 15° C. to about 40° C., about 20° C. to about 37° C., about 20° C. to about 45° C., about 25° C. to about 45° C., about 30° C. to about 45° C., about 31° C. to about 45° C., about 32° C. to about 45° C., about 33° C. to about 45° C., about 34° C. to about 45° C., about 35° C. to about 45° C., about 36° C. to about 45° C., about 37° C. to about 45° C., about 38° C. to about 45° C., about 39° C. to about 45° C., about 40° C. to about 45° C., about 41° C. to about 45° C., about 42° C. to about 45° C., about 43° C. to about 45° C., or about 44° C. to about 45° C., etc.).

In the present disclosure, the polymer solution further may comprise the target agent.

In some embodiments, the second hydrogel forming polymer may not comprise a DX-O(Me)-DTT.

In another aspect, the present disclosure provides a method for producing the composition may comprise: a) grafting the precursor polymer with the degradable linker to obtain the first hydrogel forming polymer and/or the second hydrogel forming polymer; and b) mixing the first hydrogel forming polymer and/or the second hydrogel forming polymer with an additional polymer (e.g., the second hydrogel forming polymer or the first hydrogel forming polymer) under a condition enabling formation of the hydrogel.

In some embodiments, the step of a), b) and c) may be carried out once or more (e.g., once, twice, three times or more). For example, the steps of a), b) and c) may be carried out once for producing macroscopic hydrogel or micronized hydrogel. In another example, the steps of a), b) and c) may be carried out three times for producing the micronized hydrogel in a macroscopic hydrogel.

In another aspect, the present disclosure provides a method for sustained release of a target agent, and the method may comprise: mixing the target agent with a composition to obtain a mixture and subjecting the mixture to a condition enabling formation of a hydrogel capable of sustained release of the target agent.

In another aspect, the present disclosure provides a method for sustained release of a target agent, and the method may comprise entrapping the target agent in the hydrogel.

In some cases, the method may comprise incubating the composition at about 1° C. to about 45° C. (e.g., about 1° C. to about 10° C., about 1° C. to about 8° C., about 1° C. to about 6° C., about 2° C. to about 6° C., about 3° C. to about 5° C., about 1° C. to about 45° C., about 2° C. to about 45° C., about 3° C. to about 45° C., about 4° C. to about 45° C., about 6° C. to about 45° C., about 8° C. to about 45° C., about 10° C. to about 45° C., about 15° C. to about 45° C., about 15° C. to about 40° C., about 20° C. to about 37° C., about 20° C. to about 45° C., about 25° C. to about 45° C., about 30° C. to about 45° C., about 31° C. to about 45° C., about 32° C. to about 45° C., about 33° C. to about 45° C., about 34° C. to about 45° C., about 35° C. to about 45° C., about 36° C. to about 45° C., about 37° C. to about 45° C., about 38° C. to about 45° C., about 39° C. to about 45° C., about 40° C. to about 45° C., about 41° C. to about 45° C., about 42° C. to about 45° C., about 43° C. to about 45° C., or about 44° C. to about 45° C., etc.).

In some embodiments, the method may comprise incubating the composition at about 1° C. to about 45° C. (e.g., at about 1° C. to about 10° C., at about 1° C. to about 8° C., at about 1° C. to about 6° C., at about 2° C. to about 6° C., at about 3° C. to about 5° C., at about 1° C. to about 15° C., at about 1° C. to about 20° C., at about 1° C. to about 30° C., at about 1° C. to about 40° C., at about 32° C. to about 40° C., at about 35° C. to about 40° C., such as at about 37° C.).

In another aspect, the present disclosure provides a kit, and the kit may comprise: a) the composition; and b) a target agent to be sustained released by a hydrogel formed with the composition of a).

In some cases, the kit may further comprise one or more of the following: a stabilizer, a bulking agent, a filler, a diluent, an anti-adherent, a binder, a coating agent, a coloring agent, a disintegrant, a flavor, a fragrance, a lubricant, and/or an antioxidant.

In another aspect, the present disclosure provides a use of the composition for making a hydrogel.

In another aspect, the present disclosure provides a use of the composition or the hydrogel for sustained release of a target agent.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### EXAMPLES

The following examples are set forth so as to provide those of ordinary skills in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Example 1 Conjugating Vinyl Sulfone (VS) and Thiol (SH) Groups to Dextran or Hyaluronic Acid Via Non-Hydrolysable Linkers

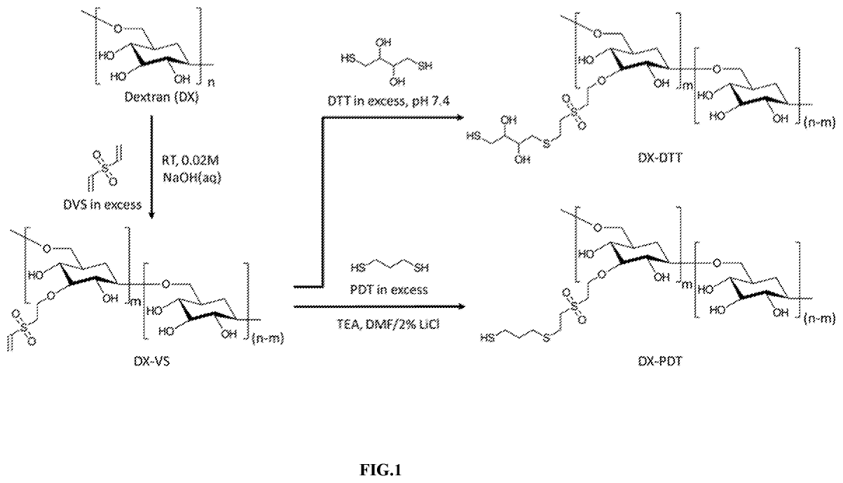

Dextran (DX) and hyaluronic acid (HA) were functionalized with vinyl sulfone (VS) and thiol (SH) using previously reported method (refers to Y. Yu and Y. Chau, “One-step ‘click’ method for generating vinyl sulfone groups on hydroxyl-containing water-soluble polymers,” Biomacromolecules, vol. 13, pp. 937-942, 2012). In brief, dextran of three molecular weights, the 150 kDa (Wako), 40 kDa (Sigma) and 6 kDa (Sigma), or hyaluronic acid of 29 kDa and 150 kDa, were grafted with VS pendant groups by reacting excess (1.2-1.5 eq to hydroxyls) divinyl sulfone (DVS, 97% contains <650 ppm hydroquinone as inhibitor, Aldrich) to the hydroxyl groups in 0.02M sodium hydroxide solution (for DX) and 0.1 M sodium hydroxide solution (for HA) with stir mixing (FIG. 1). The reaction was stopped by adding concentrated HCl to decrease the reaction pH below 5, and degree of VS modification was controlled by reaction time. The products were purified by dialysis (Spectra/Por™ cellulose membrane, 7kD MWCO, Spectrum) against deionized water under ambient temperature to remove the excess DVS and lyophilized afterwards. The lyophilized product was either stored under −20° C. upon use. Degree of modification (DM) was calculated as the number of VS groups per pyranose units of dextran or per disaccharide unit for HA. The DM of VS groups was estimated from the ¹H NMR spectroscopy with residual internal HDO (δ 4.75, 300 MHz). Relative amount of vinyl protons: δ 6.27-6.44 (q, 2H, ═CH₂), δ 6.82-6.97 (m, 1H, —CH═); relative amount of pyranose: δ 4.87-5.29 (m, 1H on Ci); relative amount of disaccharide: δ 2.0 (m, 3H, —CH₃).

Non-hydrolysable DX-SH were synthesized by reacting thiol donors varied in hydrophobicity, namely the dithiothreitol (DTT, 99%, J&K), 1,3-propanedithiol (PDT, 99%, Sigma-Aldrich) to DX-VS. For DTT conjugation, DX-VS was dissolved in 0.1M phosphate buffer (pH7.4), and purged with nitrogen gas to remove the dissolved oxygen. The DTT was dissolved in water, then added to DX-VS solution in excess (6 eq) to VS groups and reacted for two hours under ambient temperature with stir mixing. The reaction was stopped by lowering pH to 3 using (1 M) dilute hydrochloric acid. The excess DTT was removed by dialysis (7kD MWCO) against dilute HCl solution in deionized water (pH=3), then dried by lyophilization. For PDT conjugation, DX-VS were dissolved in dimethylformamide with 2% lithium chloride (DMF/2% LiCl) in 90° C. oil bath, then cooled down to ambient temperature, purged with nitrogen. PDT were added in excess (6 eq) to VS, triethylamine (TEA, 99%, Sigma-Aldrich) was added as catalyst (0.5 eq to VS). The mixture was reacted for one hour, products were precipitated in isopropanol, the pellet was resuspended in water and further purified by dialysis as described for DX-DTT.

Complete reaction of VS was confirmed by disappearance of VS related peaks in ¹H NMR spectra. Actual DM of thiol groups on DX-SH was measured by Ellman's assay (refer to Yu Y, Chau Y. “Formulation of in situ chemically cross-linked hydrogel depots for protein release: from the blob model perspective”. Biomacromolecules. 2015; 16(1):56-65).

### Example 2 Conjugating Thiols (SH) to Dextran with Hydrolysable Ester Linkers

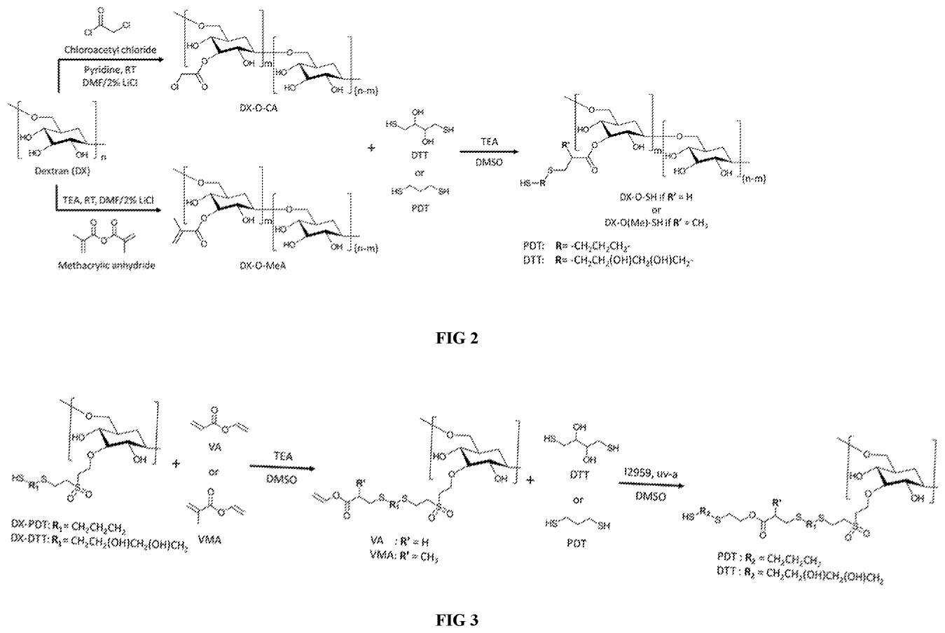

2.1 Synthesis of acrylate functionalized dextran via ester linkage (DX-O-CA) Dextran functionalized with chloroacetyl groups (DX-O-CA) was synthesized according to Ramirez's method (FIG. 2, refer to Ramirez J C, Sanchez-Chaves M, Arranz F., “Functionalization of dextran with chloroacetate groups: immobilization of bioactive carboxylic acids”. Polymer (Guildf). 1994; 35(12):2651-2655. doi:10.1016/0032-3861(94)90394-8). In brief, dextran (40 kDa) was dissolved in DMF/2% LiCl at 90° C. oil bath. Dextran solution was cooled down to ambient temperature, then pyridine (99%, VWR Chemicals BDH) was added to the solution (1 eq to the OH of dextran). Chloroacetyl chloride (99%, Sigma) was added (0.1-0.5 eq to OH of dextran) to react for 2-6 hours. DM can be controlled by the amount of chloroacetyl chloride and reaction time. Product DX-O-CA was purified by reprecipitation in isopropanol and dried in vacuum. The DM of acrylate was quantified using 1H NMR: chloroacetate: S 4.29-4.37 (m, 2H, —CH₂—).

2.2 Conjugation of Thiol Donors to DX-O-CA

Dried DX-O-CA were dissolved in 0.5M phosphate buffer (pH7.4), and then purged with nitrogen gas. DTT aqueous solution (6˜10 eq to CA) was added to DX-O-CA and reacted for two hours under ambient temperature (FIG. 2). The reaction was stopped by adding dilute hydrochloric acid to decrease the reaction pH to 4. Excess DTT was removed by dialysis (7kD MWCO) against deionized water, then dried by lyophilization. The DM of thiol was quantified using Ellman's assay.

2.3 Synthesis of Methacrylate Functionalized Dextran Via Ester Linkage (DX-O-MeA)

Methacrylate was conjugated to dextran via oxy-ester linkage according to Kim and Chu's protocol (FIG. 2). In brief, dextran (150 kDa or 40 kDa) were dissolved in DMF/2% LiCl (5 w/v %) at 90° C. oil bath, then cooled down to ambient temperature. Methacrylate anhydride (MA, 94%, Aldrich) was added (0.3˜0.5X to pyranose), and the catalyst TEA was added (0.01-0.1 eq to MA). Reaction was proceeded under ambient temperature for overnight with stir mixing. Intermediate dextran-methacrylate (DX-O-MeA) were precipitated using isopropanol for three times, the pellet was dried in vacuum. The dried pellet was resuspended in water, further purified by dialysis (7kD MWCO) against deionized water and lyophilized. The DM of methacrylate was quantified using ¹H NMR spectroscopy: vinyl protons: S 5.71-6.20 (d, 2H, ═CH₂), methyl protons: δ 1.9 (m, 3H, —CH₃).

2.4 Conjugation of Thiol Donors to DX-O-MeA

Lyophilized DX-O-MeA was dissolved in DMSO at 2˜5% w/v and purged with nitrogen gas. Four types of thiol donors varied in hydrophobicity: 1,2-ethanedithiol (EDT, 98%, Sigma-Aldrich); 1,3-propanedithiol (PDT, 99%, Sigma-Aldrich); 2,3-dimercapto-1-propanol (DMP, 98%, Sigma-Aldrich); and DTT were conjugated to the DX-O-MeA via TEA (0.5 eq to MA) catalyzed Michael addition. The thiol donors were added in excess (6-10 eq to MA), and reacted for one hour at ambient temperature with stir mixing (FIG. 2). Thiolated dextran were collected and purified using the same method for DX-O-MeA. The complete consumption of MA was confirmed by the disappearance related signals in the ¹H NMR spectra. The DM of thiol groups was quantified by Ellman's assay.

2.5 Conjugation of Vinyl Acrylate (VA) and Vinyl Methacrylate (VMA) on Dextran

The vinyl acrylate (VA) and vinyl methacrylate (VMA) were conjugated to dextran as shown in FIG. 3. Non-hydrolysable DX-DTT, or DX-PDT (obtained from example 1) were dissolved in dimethyl sulfoxide (DMSO, 99%, Sigma-Aldrich) at 2˜5% w/v and purged with nitrogen. Vinyl acrylate (VA, 98%, Sigma-Aldrich), or vinyl methacrylate (VMA, 98%, Sigma-Aldrich) were added in excess (10˜20 eq to SH). TEA was added as a catalyst at final concentration of 0.5% v/v. Reaction was conducted under ambient temperature for one hour with stir mixing. The polymers were precipitated in isopropanol, the pellets were briefly dried in vacuum, and redissolved in deionized water and further purified by dialysis (7kD MWCO) against deionized water, and then dried by lyophilization. The DM of vinyl was quantified using ¹H NMR spectroscopy: vinyl protons: δ 7.10-7.22 (dd, 1H). These two polymers were denoted as DX-SH-VA and DX-SH-VMA.

2.6 Conjugation of Thiol Groups on DX-SH-VA and DX-SH-VMA

Dried DX-SH-VA or DX-SH-VMA were dissolved in DMOS at 2˜5% w/v and purged with nitrogen gas. Radical initiator I-2959 (Irgacure-2959, 98% Sigma-Aldrich) were added at final concentration of 0.5 w/v %. Thiol donors (PDT or DTT) were added in excess (10 eq to vinyl) and conjugated to the vinyl group by radical thiol-ene addition. Reaction was proceeded in quartz tube under UV-A (354 nm) illumination for 3 hours at ambient temperature with stir mixing. Final products were purified by precipitation and dialysis, and freeze dried similar to previous examples. The DM of thiol was quantified using Ellman's assay.

The obtained modified dextran with different hydrolysable ester linkers were shown in Table 1.

TABLE 1
Chemistry of selected ester linkers

| Ester linker structure | | Abbreviation |
|---|---|---|
| S1 | 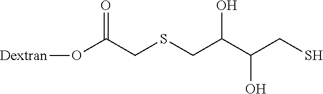 | DX-O-DTT |
| S2 | 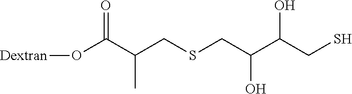 | DX-O(Me)-DTT |
| S3 | 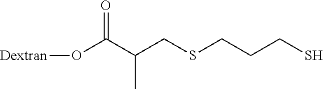 | DX-O(Me)-PDT |
| L1 | 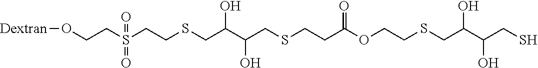 | DX-DTT-VA-DTT |
| L2 | 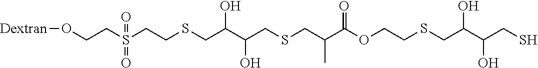 | DX-DTT-VMA-DTT |
| L3 | 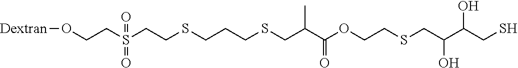 | DX-PDT-VMA-DTT |
| L4 | 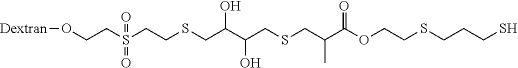 | DX-DTT-VMA-PDT |

The polymers were abbreviated in format of [polymer, molecular weight, functional group, DM]. For example, the VS modified dextran with 40 kDa and 5% DM was denoted as DX40k-VS_5 and DX40k-DTT_5. The -SH functionalized dextran with an easter linker are abbreviated “DX-O-SH”.

### Example 3 Synthesis of Maleimide Modified Hyaluronic Acid (HA-MI) Containing a Hydrolytic Group

Hyaluronic acid (HA) with molecular weight 27 kDa was obtained from Contipro a.s (Dolni Dobrouc, Czech Republic). A molecule contains maleimide group (MI molecule) (see structure:

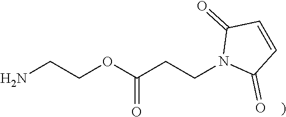 [SMILES: NCCOC(=O)CCN1C(=O)C=CC1=O]

was provided by the contracted research organization South University of Science and Technology of China. 4-(4,6-Dimethoxy-1,3,5-triazin-2-yl)-4-methylmorpholinium chloride (DMTMM) was obtained from Aladdin Biotechnology. Non-hydrolysable thiol modified dextran (DX-SH) was synthesized as in Example 1.

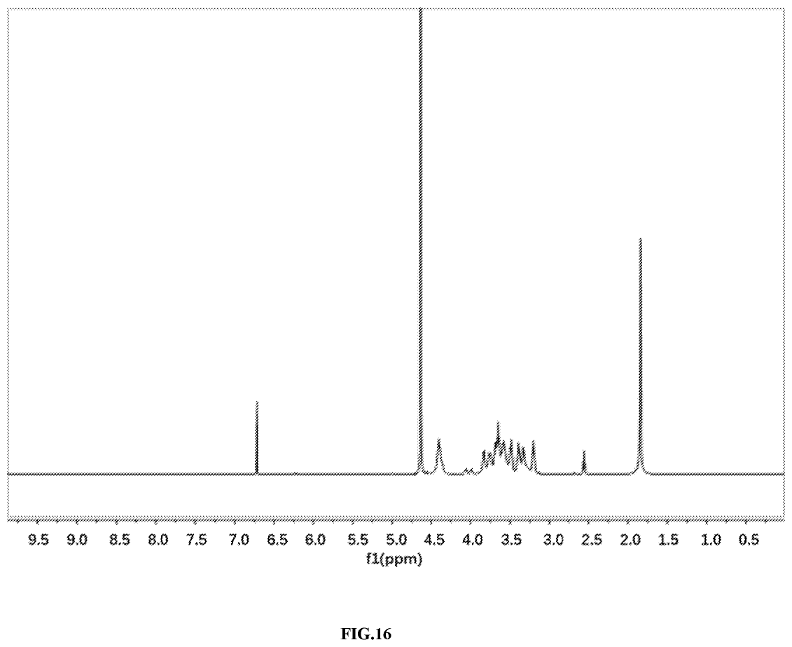

27 kDa HA was dissolved in 1 mM PB at concentration of 24 mg/ml. MI molecule was dissolved in 1 mM PB at concentration of 9.72 mg/ml. After completed dissolution, equal amount of HA solution and MI solution was mixed in a 20 ml glass scintillation vial by stirring with 2 ml each. pH value was then adjusted by dropwise addition of 400ul or 800 μL of 0.1 M NaOH solution before the addition of 66.4 mg of DMTMM. The molar ratio of —COOH from HA to —NH₂ from MI to DMTMM was 1:0.5:2. The reaction was stopped in 72 h by addition of 160 μL of 25% NaCl and precipitation in 20 mL ethanol in a 50 mL conical tube. The precipitate was separated via centrifugation at 8000 rpm for 5 min and decanting of the supernatant liquid. The residue pellet was re-dissolved in 10 mL of DI and further purified by dialysis in in 4 L 0.6 mM HCl solution (pH=4) for three days. The dialysis buffer was changed twice a day. A white cotton like solid was obtained after lyophilization for 2 days. The structure of the product was characterized by ¹H NMR. The results are shown in FIG. 16. The HA-MI was synthesized successfully.

### Example 4 Preparation of Hydrogels

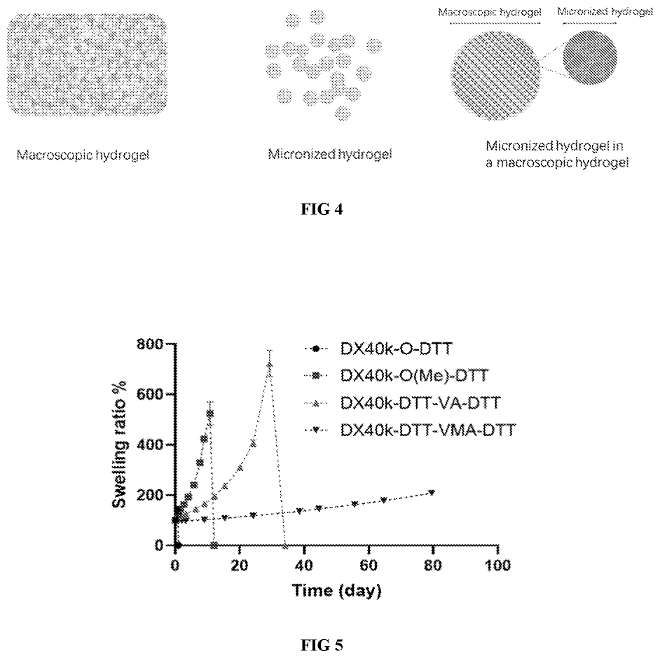

The hydrogel can be of three forms, macroscopic hydrogel, micronized hydrogel, or micronized hydrogel in a macroscopic hydrogel (FIG. 4).

4.1 Preparation of Blank and Protein Laden Hydrogels

Blank hydrogels were formed by mixing different -VS and -SH functionalized polymers at 1:1 volume ratio. The -VS functionalized hydrogel precursors (DX-VS) were dissolved in pH 7 PBS. The thiol functionalized polymers (DX-DTT, and hydrolysable DX-O-SH) were dissolved in water to minimize disulfide crosslinking during dissolution. The precursor polymers were mixed thoroughly at 4° C., and pipetted on a hydrophobic surface as hemispherical droplets of about 30-50ul, then incubated in a humid chamber at ambient temperature for overnight. The wet weight of hemispherical hydrogels at relax state was defined as initial weight W₀. Two types of IgG protein, bevacizumab (Avastin® Roche Ltd, Basel, Switzerland) and IgG-FITC (from human serum, Sigma-Aldrich), were used. Protein loaded hydrogels were formed using the same method as blank hydrogel, except dissolving the VS polymers in pH adjusted protein solutions (pH=7), and unless specified, the -VS polymers were mixed with -SH polymers at 1:2 mass ratio to minimize the undesired reaction between laden proteins with remaining VS groups. Unless specified, for the formulations used for in vivo application, HA-VS was dissolved in pH adjusted Avastin solutions, of which the pH was about 7 by adding with 1/10 volume of the 0.4M Na₂HPO₄ buffer. DX-SH was dissolved in Avastin solution directly.

4.2 Preparation of Micronized Hydrogel

The dissolved HA-VS and DX-SH were dissolved in Avastin solution as described in Example 3.1 and mixed thoroughly. About 400 μL was transferred into 20 mL oil phase, and stirred using ordinary vortex at max speed for one hour under ambient temperature form the micronized hydrogels (microgel). The oil phase was a mixture of SPAN-80/TWEEN-80/n-heptane at volume ratio of 2:1:97. After brief spin down of the microgels, the supernatant oil phase was discarded. The microgels were sequentially washed with excess absolute ethanol and DI water, each for 6 times. Microgels were collected after each washing step using centrifugation below 5000 rpm. Afterwards, Avastin was added to the microgels and stored at 4° C. upon use.

Alternatively, the particle can be made by a microfluidic device.

4.3 Preparation of Micronized Hydrogel in a Macroscopic Hydrogel

The HA-VS was dissolved in 0.1M phosphate buffer (pH7.4), and the DX-SH was dissolved DI water. The two components were mixed thoroughly and temporally stored in ice. This mixture would be used as the macrogel precursor. The microgel prepared according to Example 3.2 was transferred to a centrifuge tube. The excess Avastin solution was removed by pipetting and the microgels were weighed. The macrogel precursor in liquid form was added into the microgels at 1:1 weight ratio and mixed. For in vivo study, the mixture was injected into the rabbit vitreous chamber. For in vitro release and degradation/swelling study, the mixture was pipetted on a hydrophobic surface as hemispherical droplets of about 30-50ul, and then incubated in a humid chamber at ambient temperature for overnight.

### Example 5 Measurement of Swelling and Degradation of Hydrogel

Hydrogel was placed in a 2 ml centrifuge tube and 1 ml PBS with 0.02w/v % NaN₃ were used as swelling buffer, and incubated at 37° C. At predetermined time point, the hydrogel was taken out from the swelling buffer, carefully blotted dry using a tissue paper, and weighted. The swelling ratio (Q_{w}) of hydrogels was defined as the wet weight at time t (Wₜ) over the weight of hydrogel before swelling (W₀).

### Example 6 Measurement of Hydrolysis Kinetics of Hydrolysable Polymer

The ester hydrolysis kinetics of these hydrolysable hydrogel precursors were measured using ¹H NMR in D₂O as described earlier (refer to Lau C M L, Jahanmir G, Chau Y. “Local environment-dependent kinetics of ester hydrolysis revealed by direct ¹H NMR measurement of degrading hydrogels”. Acta Biomater. October 2019). In brief, sample polymers were dissolved in 0.2M phosphate buffer (pD7.7) prepared using D₂O (99.8 atom % D, J&K) as solvent, and incubated under 37° C. The ¹H NMR spectra were recorded periodically using VNMRJ 2.2D (Agilent, US) on a Varian mercury 300 MHz high resolution NMR spectrometer. Ester hydrolysis rate were calculated from the change of the ester neighbouring methyl integrals.

The online platform Chemicalize developed by ChemAxon (https://chemicalize.com/) was used for prediction of pKa values.

### Example 7 Controlling Hydrogel Degradation by Modulating Ester Linker Chemistry

The structures of different dextran conjugated ester linkers, as well as the calculated hydrolytic half-life were summarized and compared (Table 2). The t_{0.5} of esters were measured based on the characteristic chemical shift of ester neighbouring methyl group in ¹H NMR spectra in pD7.7 phosphate buffer prepared using D₂O as solvent.

TABLE 2
The calculated hydrolysis half-life of ester linkers

| Polymer | | t_{0.5} | |
|---|---|---|---|
| S1 | DX-O-DTT | Not measurable | |
| S2 | DX-O(Me)-DTT | 5.6 | days |
| S3 | DX-O(Me)-PDT | 7.4 | days |
| L1 | DX-DTT-VA-DTT | Not measurable | |
| L2 | DX-DTT-VMA-DTT | 54 | days |
| L3 | DX-PDT-VMA-DTT | 113 | days |
| L4 | DX-DTT-VMA-PDT | 53 | days |

The DX-O-DTT had a simple ester chemistry an ester was directly conjugate to the dextran pyranose. The DX-O(Me)-DTT differs from DX-O-DTT by one more carbon and a methyl group next to the carbonyl. The increase and hydrophobicity and the electron donating effect of the methyl group increased the degradation time from 8 hours to 2 weeks (FIG. 5). To further increase the hydrolytic half-life, a more hydrophobic thiol donor PDT was used instead of DTT. The increase in the hydrophobicity further prolonged the ester t_{0.5} from about 5.6 days to about 7.4 days (Table 2).

The above-mentioned hydrogel formulations all degraded within several weeks, which were too short for most controlled release applications. We attributed the result to the limitation that only the carbonyl-side was modified in above mentioned ester linkers, which only affected the carbonyl susceptibility to OH—. However, the leaving propensity of the alkyloxy side was not altered. Given the strongest acidic pKa of the dextran hydroxy groups is 11.8 at 37° C. (refer to Larsen C. “Macromolecular prodrugs. XIII. Determination of the ionization constant of dextran by potentiometric titration and from kinetic analysis of the hydrolysis of dextran indomethacin ester conjugates”. Int J Pharm. 1989; 52(1):55-61), any modification, for example conjugating an electron withdrawing neighbouring group that increases the hydroxy pKa would prolong the half-life, and vice versa. According to this principle, two leaving groups, 1-(hydroxymethylthio)-4-mercapto-2,3-butanediol and the (3-mercaptopropylthio) methanol, which has a pKa value of 15.8 and 15.6 respectively were designed.

Experimentally measured half-life of DX-DTT-VMA-DTT was about 54 d, and 53 d for the DX-DTT-VMA-PDT. The results were well aligned with the general understanding of the reaction mechanism: a worse leaving group (R—OH) would prolong the ester half-life. In addition, the hydrophobic modulator on the carbonyl side exhibited a synergistic effect when the alkyloxy side was stabilized with DTT, as the half-life of DX-PDT-VMIA-DTT was about 113 d (table 2).

### Example 8 Prevention of Protein Covalent Binding to the Polymer Network

### 8.1 Protein Integrity Analysis by Native SDS-PAGE

The IgG laden (F-IgG and bevacizumab) hydrogels of table 3 were obtained as described in example 3.1 with various VS polymer/SH polymer ratio.

The hydrogels were placed in a 2 ml tube and 1 ml PBS was added to the gel. The tube was then incubated at 37° C. until the gel is totally degraded. Non-reducing SDS-PAGE was performed for the degradation product to evaluate the MW of protein after degradation. The F-IgG in PBS and F-IgG dissolved 30% DX-VS of 5% DM, as well as Avastin and DX-VS of 5% DM dissolved in Avastin at 30% were used as controls. The SDS-PAGE experiment was conducted using precast 4-15% gradient gel (BeyoGel Plus PAGE, Beyotime, China) with Mini-PROTEAN System (Bio-Rad Laboratories, USA) according to manufacturer's guideline. The protein was stained with Coomassie Blue (BeyoBlue, Beyotime, China) (or imaged with UV mode for FITC-IgG, F-IgG) with reference to prestained protein ladder (BeyoColor 6.5-270 kDa, Beyotime, China). To quantify the percentage of free protein in the gel, the band intensity of released IgG was compared to the band intensity of the non-encapsulated IgG protein using ImageJ 1.52 according to the online tutorial (https://di.uq.edu.au/community-and-alumni/sparq-ed/sparq-ed-services/using-imagej-quantify-blots).

### 8.2 Result

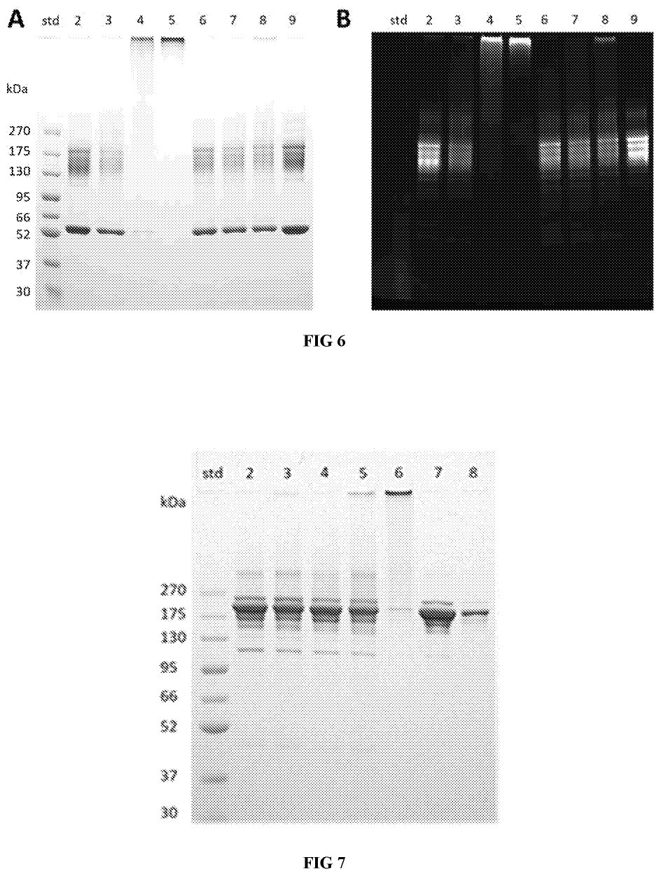

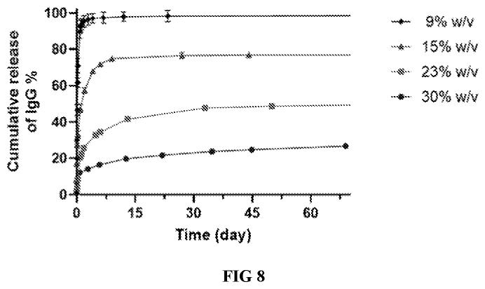

Non-reducing SDS-PAGE was conducted to evaluate the protein size after being released form the completely degraded hydrogels (FIG. 6-7). The hydrolysable hydrogels were prepared by mixing DX40k-VS and DX40k-O(Me)-DTT, both having a DM of 5%, at different concentrations (Table 3 and FIG. 6). The loading of F-IgG and incubation was the same as described previously. After all hydrogels were completely degraded, the crude mixture of F-IgG and degradation products were subjected to non-reducing SDS-PAGE analysis without purification. The native FI-IgG and the F-IgG with native DX40k were included as control. The PAGE gel was imaged under brightfield (FIG. 6A) and UV respectively (FIG. 6B). Most of the proteins were trapped in the well when the VS polymer/SH polymer mass ratio (hereinafter refer to as VS/SH ratio) is higher than 0.67 from the chemical conjugation to hydrogel precursors. Decreasing the VS/SH ratio to 0.67 was effective to inhibit the undesired VS-amine binding and preserve the laden proteins in their native conformation, as thiols have much higher reaction selectivity to vinyl sulfones than the amines. Since the commercially available F-IgG was polyclonal, and was added with BSA as the stabilizer (not mentioned in product description, but clarified by the technical support), multiple bands were observed in the SDS-PAGE gel.

The monoclonal antibody bevacizumab released from hydrogels composed of DX40k-VS and DX40k-O(Me)-DTT, both having a DM of 5%, at different concentrations was analyzed using the same method. The result is similar, in order for protein not to be bound to the polymer, the VS/SH ratio should be lower than 1 (Table 4 and FIG. 7). Comparing to Lane 7, the amount of free protein in Lane 2 to Lane 6 was 99.1%, 97%, 98.9%,90.7%, 120.3% accordingly.

TABLE 3
Sample formulations

| | | | Conc. of | | |
|---|---|---|---|---|---|
| | | Conc. of | DX40k- | Total | |
| | | DX40k-VS | O(Me)-DTT | polymer conc. | |
| | | in the hydrogel | in the hydrogel | in the hydrogel | VS/SH |
| Lane | Sample | w/v % | w/v % | w/v % | ratio |
| 2 | F-IgG + DX40k | — | — | — | — |
| | solution | | | | |
| 3 | Released F-IgG with | 8% | 12% | 20% | 0.67 |
| 4 | hydrogel degradation | 10% | 10% | 20% | 1 |
| 5 | product | 12% | 8% | 20% | 1.5 |
| 6 | | 5% | 10% | 15% | 0.5 |
| 7 | | 7.5% | 15% | 22.5% | 0.5 |
| 8 | | 10% | 20% | 30% | 0.5 |
| 9 | F-IgG only | — | — | — | — |

TABLE 4
Sample formulations

| | | | Conc. of | | |
|---|---|---|---|---|---|
| | | Conc. of | DX40k- | | |
| | | DX40k-VS | O(Me)-DTT | Polymer conc. | |
| | | in the hydrogel | in the hydrogel | in the hydrogel | VS/SH |
| Lane | Sample | w/v % | w/v % | w/v % | ratio |
| 2 | Total bevacizumab release | 5% | 10% | 15 | 0.5 |
| 3 | from degraded hydrogel | 10% | 20% | 30 | 0.5 |
| 4 | | 6 | 9 | 15 | 0.67 |
| 5 | | 12 | 18 | 30 | 0.67 |
| 6 | | 15 | 15 | 30 | 1 |
| 7 | Bevacizumab + DX40k | — | — | — | — |
| | solution | | | | |
| 8 | Bevacizumab | — | — | — | — |

### Example 9 Measurement of Protein Release from Hydrogel Depot

Hydrogels were placed in a 2 ml or 4 ml tubes and 1 ml PBS with 0.02w/v % NaN₃ were used as releasing buffer. The NaN₃ was added to prevent bacteria growth in the releasing buffer during the long-term incubation. Unless specified, the pH for the PBS was 7.4. In some cases, the pH was adjusted to 4.5. At each time point, the releasing buffer was taken out and replaced with fresh buffer. The concentration of bevacizumab in the releasing buffer was measured by Bradford's Assay (Bio-Rad Laboratories, Inc, California, USA) according to the manufacturer's instruction. The concentration of F-IgG was measured by spectrophotometry at 490/520 nm excitation/emission using 96-well plate. The fluorescence intensity—IgG concentration standard curves were established at pH 4.5 and pH 7.4 PBS respectively.

### Example 10 Controlling Initial Release by Manipulating Hydrogel Mesh Size

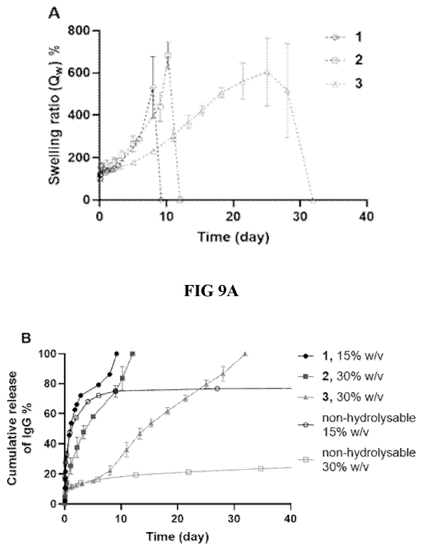

The average mesh size (ξ_{avg}), and its polydispersity of a hydrogel are considered to be a key parameter governing the diffusion behavior of solute molecules within a polymer meshwork in theory. The cumulative release of the model protein bevacizumab from non-hydrolysable hydrogels varying polymer concentrations was probed to demonstrate the relationship between initial release and ξ_{avg}. The ξ_{avg} was adjusted via altering polymer concentration at relax state only (table 5). Molecular weight and DM were kept the same across different groups. By increasing polymer concentration from 9% to 30% w/v, the fraction of initial release in the first day was controlled from 90% to only 10% (FIG. 8).

TABLE 5
Summary of non-hydrolysable dextran hydrogel formulations

| Conc. % w/v of total | Conc. % w/v of DX40k- | Conc. % w/v of DX40k- |
|---|---|---|
| polymer in the hydrogel | VS in the hydrogel | DTT_5 in the hydrogel |
| 9 | 3 | 6 |
| 15 | 5 | 10 |
| 23 | 8 | 15 |
| 30 | 10 | 20 |

### Example 11 Sustained Protein Release by Crosslink Degradation

The protein release behavior consists of two phases. For the initial phase, the protein was released from the hydrogel and the release rate was related to the polymer concentration. A second phase where the protein was not able to be released or release with a very slow rate from the gel was seen in all hydrogel formulation.

Hydrolysable gel using DX40k-O(Me)-DTT to crosslink with DX-VS were synthesized as described in example 3. The formulation of polymer concentration and VS/SH ratio were showed in table 6. F-IgG was used as the model protein in all hydrogels.

TABLE 6
Hydrogel formulations

| | | | | | Total | |
|---|---|---|---|---|---|---|
| | | | | Polymer | polymer | |
| | | | Modifi- | Conc. | conc. | |
| | | Molecular | cation | w/v % | in the | VS/ |
| Hydro- | | weight of | degree | in the | hydrogel | SH |
| gel | Formulation | Dextran | (%) | hydrogel | w/v % | ratio |
| 1 | DX-VS | 40 k | 5 | 5 | 15 | 0.5 |
| | DX-O(Me)- | 40 k | 5 | 15 | | |
| | DTT | | | | | |
| 2 | DX-VS | 40 k | 5 | 10 | 30 | |
| | DX-O(Me)- | 40 k | 5 | 20 | | |
| | DTT | | | | | |
| 3 | DX-VS | 150 k | 8 | 10 | 30 | |
| | DX-O(Me)- | 40 k | 8 | 20 | | |
| | DTT | | | | | |

The ester in DX-O(Me)-DTT has a hydrolytic half-life (37° C. at pH 7.4) about 5.6 d at the solution state (Table 6), and 2.9 d at the hydrogel state when crosslinked with DX-VS (refer to au CML, Jahanmir G, Chau Y. “Local environment-dependent kinetics of ester hydrolysis revealed by direct 1H NMR measurement of degrading hydrogels”. Acta Biomater. October 2019).

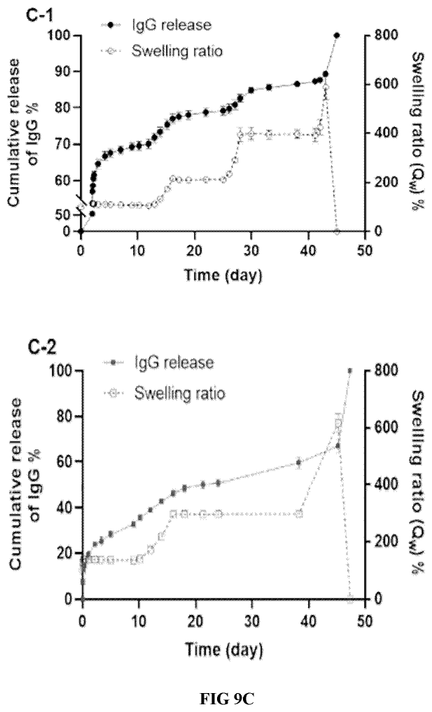

Since the polymer matrix of a hydrogel is highly hydrated, the hydrolytic cleavage rate is expected to be ubiquitous over the entire hydrogel. Therefore, the hydrolysable hydrogels predominantly degrade in bulk rather than on the surface. Random cleavages of the esters at the crosslinks lead to a decrease in effective number of crosslinks. According to Flory's model (refer to Peppas N A, Lustig S R. Solute Diffusion in Hydrophilic Network Structures. In: Peppas N A, ed. Hydrogels in Medicine and Pharmacy. Vol. 1. Fundamentals. Boca Raton, Fla.: CRC Press; 1986:57-83), a decrease in the number of crosslink will reduce the elastic energy of the gel and the swelling ratio will increase accordingly. Therefore, the change of swelling ratio over time was used as an indicator bulk erosion (FIG. 9A).

In all formulation, a portion of protein was not releasable or release at an almost undetectable rate for non-degradable gel (FIG. 8), but all protein were releasable in hydrogels containing hydrolytic crosslinks (FIG. 9B). When the DM and molecular weight of precursor polymers were kept constant, increasing the total polymer concentration from 15% to 30% w/v reduced the initial release and a smoother, degradation driven release profile can be obtained (FIG. 9B).

When the total polymer concentration was kept at 30% w/v, increasing the DM from 5% to 8%, and the molecular weight of the DX-VS from 40 kDa to 150 kDa prolonged the gel life from 12 days to more than 30 days (FIG. 9A).

The release behavior of laden IgG was similar between degradable and non-hydrolysable hydrogel in the initial stage, and the release curves diverged afterwards. The IgG molecules were gradually released from the hydrolysable hydrogels until the meshwork was completely disintegrated, while the IgG release rate was very low for non-hydrolysable hydrogels (FIG. 9B).

The relation between hydrogel degradation and protein release by varying the pH of releasing buffer were further investigated. A decrease in pH from 7.4 to 4.5 in the releasing buffer is expected suppress the OH— catalyzed hydrolytic cleavage, which can be reflected from a change in the rate of swelling. We can see that except for the initial release phase, the release rate of protein was significantly lowered in pH 4.5, but significantly accelerated in pH 7.4.

Shifting the pH alternatively between 7.4 and 4.5 led to a fast/slow release pattern of IgG in response to the pH change.

These data suggested the IgG release was degradation dependent.

### Example 12 Controlling the Protein Release Rate by Mixing of Different Hydrolytic Backbones

Two hydrolysable hydrogel formulations, the fast degrading component A (linker S2, t_{0.5}=5.6d) and the slow degrading component B (linker L2, t_{0.5}=54d) were made as described in example 3. The formulation was shown in table 7.

TABLE 7
Hydrogel formulations

| | | | | Polymer | | |
|---|---|---|---|---|---|---|
| | | | Modifi- | Conc. | Total | |
| | | Molecular | cation | w/v % | polymer | VS/ |
| Hydro- | | weight of | degree | in the | conc. | SH |
| gel | Formulation | Dextran | (%) | hydrogel | w/v % | ratio |
| A | DX-VS | 40 k | 5 | 10 | 30 | 0.5 |
| | DX-O(Me)- | 40 k | 5 | 20 | | |
| | DTT | | | | | |
| B | DX-VS | 40 k | 5 | 10 | | |
| | DX-DTT- | 40 k | 5 | 20 | | |
| | VMA-DTT | | | | | |
| 50% A + | DX-VS | 40 k | 5 | 10 | | |
| 50% B | DX-O(Me)- | 40 k | 5 | 10 | | |
| | DTT | | | | | |
| | DX-DTT- | 40 k | 5 | 10 | | |
| | VMA-DTT | | | | | |
| 25% A + | DX-VS | 40 k | 5 | 10 | | |
| 75% B | DX-O(Me)- | 40 k | 5 | 5 | | |
| | DTT | | | | | |
| | DX-DTT- | 40 k | 5 | 15 | | |
| | VMA-DTT | | | | | |

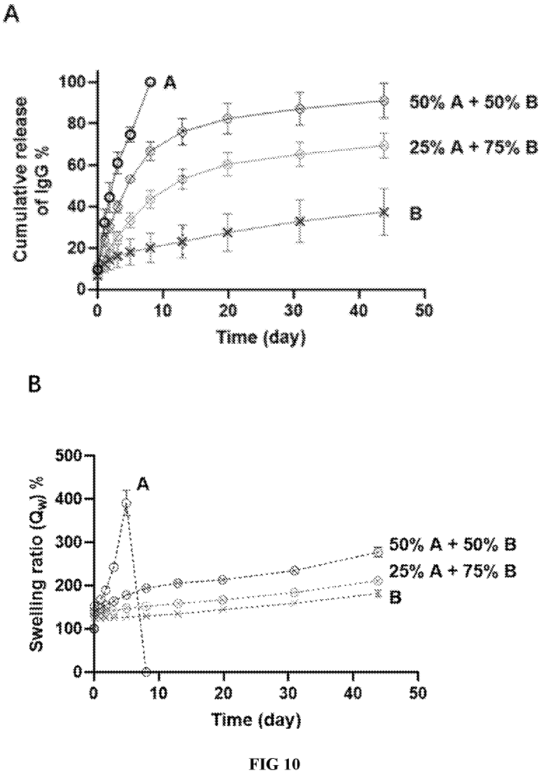

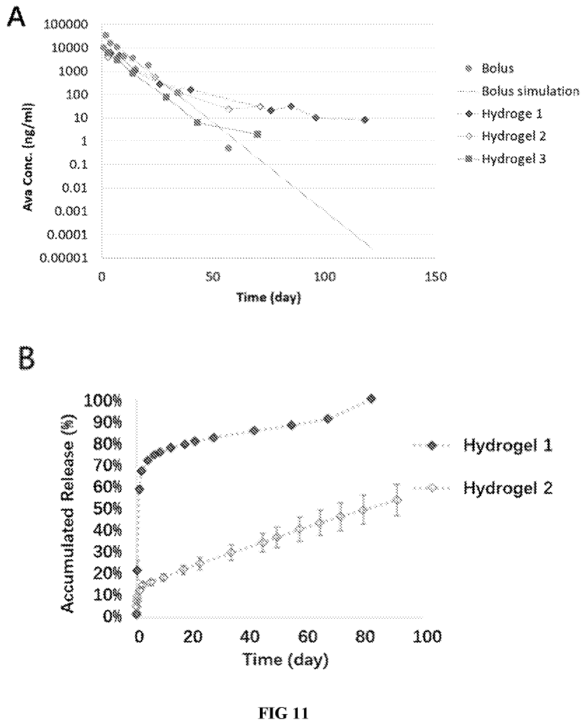

All other formulation parameters were kept the same. Polymer concentration was controlled at 30% w/v, and the VS/SH ratio was at 0.5. The component A and B were mixed at two ratios: 25A/75B, and 50A/50B to yield two hybrid hydrogels. FIG. 10 showed the cumulative release of F-IgG (A) and corresponding hydrogel swelling (B) at 37° C., pH 7.4. Data are presented as mean±SD (n=3).

### Example 13 In Vivo Pharmacokinetics of Protein-Encapsulating Hydrogels

Hydrogel 1, 2 and 3 were obtained as described in example 3. Hydrogel 1 composed of HA-VS and DX-DTT-VMA-DTT at polymer mass ratio of 1:2 and a total polymer concentration of 23%. Hydrogel 2 composed of micronized hydrogel in a macroscopic hydrogel. The micronized hydrogel composed of HA-VS and DX-DTT-VMA-DTT at polymer mass ratio of 1:2 and a total polymer concentration of 23%, and the macroscopic hydrogel composed of HA-VS and DX-DTT-VMA-DTT at polymer mass ratio of 1:2 and a total polymer concentration of 18%. Hydrogel 3 was micronized hydrogel composed of HA-VS and DX-DTT-VMA-DTT at polymer mass ratio of 1:2 and a total polymer concentration of 23%. The formulations of Hydrogel 1, 2 and 3 were shown in table 8.

The hydrogel formulation 1 and 2 was able to release bevacizumab in vitro for at least 3 months. The in vitro release kinetics for formulation 3 was not measured because the particle may be removed by pipetting during release measurement, but it would be expected to continue to release protein similar to Formulation 1 and 2 because it is the same as the microgel in Formulation 2 (FIG. 11B).

TABLE 8
Hydrogel formulations

| | | | Molecular | Modifi- | Total | |
|---|---|---|---|---|---|---|
| | | | weight of | cation | polymer | VS/ |
| Hydro- | | | Dextran | degree | conc. | SH |
| gel | Formulation | | (Dalton) | (%) | w/v % | ratio |
| 1 | Macroscopic | HA-VS | 29 k | 8 | 23 | 0.5 |
| | hydrogel | DX-DTT- | 40 k | 4 | | |
| | | VMA-DTT | | | | |
| 2 | Micronized | HA-VS | 29 k | 11 | 23 | |
| | hydrogel | DX-DTT- | 40 k | 4 | | |
| | | VMA-DTT | | | | |
| | Macroscopic | HA-VS | 29 k | 8 | 18% | |
| | hydrogel | DX-DTT- | 40 k | 4 | | |
| | | VMA-DTT | | | | |
| 3 | Micronized | HA-VS | 29 k | 11 | 23% | |
| | hydrogel | DX-DTT- | 40 k | 4 | | |
| | | VMA-DTT | | | | |

13.1 Intravitreal Injection in Rabbit

Female New Zealand White rabbits of 3 to 4 kg were used in this study. Before all treatments, the rabbits were anesthetized with intramuscular injection of a ketamine/medetomidine cocktail. Hydrogel precursors prepared as described before, and were chilled in ice bath. After thorough mixing, then mixture was loaded into an insulin syringe with 29-gauge, 12 mm long needle. Before injection, the cornea of anesthetized rabbits was topically anesthetized with Alcaine, then disinfected with Tobrex. About 40 μL, which contained about 1 mg bevacizumab was intravitreally injected to the eye at the pars plana 3 mm behind the limbus at the superior temporal region. Tobramycin ointment was applied on the ocular surface to avoid post injection infections. The bolus injection of PBS or bevacizumab (Avastin) was conducted in the same way.

The retinal fundus, and the intravitreal hydrogels were periodically visually examined using a fundus imaging system (Volk iNview, Volk Optical, US) attached to an iPhone 6S with the operating system iOS9 (Apple, US). Before examination, the rabbits were anesthetized, and the pupil was dilated with Mydriacyl®. The superior, inferior, temporal and nasal regions near the optic disk were documented.

The IOP was measured using a tonometer (TonoVet, icare, Finland) according to the manufacturer's manual instruction. The average IOP was calculated from 6 readings for each eye at each time point.

13.2 Measurement of Protein in Rabbit Eye

At each time point, around 150 μL of aqueous humour was sampled from the anterior chamber using an insulin syringe with 31-gauge needle. The samples were diluted in equal volume of 2% w/v bovine serum albumin (BSA) in PBS, and stored in −80° C. freezer until measurement. The bevacizumab in the aqueous humour was quantified by Sandwich Enzyme-linked immunosorbent assay (ELISA) according to Yu et al. (refer to Y. Yu, X. Lin, Q. Wang, M. He, and Y. Chau, “Long-term therapeutic effect in nonhuman primate eye from a single injection of anti-VEGF controlled release hydrogel,” Bioeng. Transl. Med., 2019). Briefly, lyophilized VEGFA-165 was dissolved in water at 100ug/mL as the stock, then diluted in PBS to 0.3 ug/mL as the concentration for coating. PBS with 0.05% v/v TWEEN20 was used as washing buffer. Blocking buffer was 1% w/v BSA in PBS. Bevacizumab standards, aqueous samples and the IgG-HRP were diluted in the 1% BSA as well.

A high affinity 96-well plate was coated with 90 μL of 0.25 μg/mL Avastin/PBS at 4° C. for overnight. After blocking with 350 μL 1% BSA for 2h, bevacizumab standards and the aqueous humour samples of 100 uL were incubated for another 2h, followed by 1 h incubation of 100 μL of IgG-HRP at 1 μg/mL concentration. After each step, each well was washed with 300 μL of washing buffer for three times. Except coating, all incubation steps were conducted at ambient temperature. Afterwards, 100 uL TMB was added to each well, the incubated in dark for 15˜30 min, depends on the color intensity. After sufficient blue color was developed, the reaction was terminated by adding 50 L of 2M HCl per well, and the color was changed to yellow. The standards were measured in triplicates and aqueous samples were measured in duplicates. Absorbance at 450 nm was measured on a Varioskan LUX plate reader (ThermoFisher), and absorbance to bevacizumab concentration standard curve was fitted with using 5-parameter logistic (5PL) algorithm using SkanIt 6.0 (ThermoFisher).

13.3 Results

For bolus injection, the bevacizumab concentration decreased in the eye at a first order elimination kinetics. The calculated half-life was 4 days. In contrast, for all three hydrogels formulations, the rate of elimination was significantly reduced after about 40 days. At day 57, the aqueous concentration of bevacizumab was no longer detectable in the bolus injection group, but continued to be detectable in all hydrogel groups, demonstrating the hydrogels was able to release protein in the eye over months (FIG. 11A). The simulation of bevacizumab concentration in the eye after bolus injection was based on the first-order elimination kinetics with the calculated half-life.

### Example 14 In Vivo Biocompatibility of Protein-Encapsulating Hydrogels in Rabbit Eyes

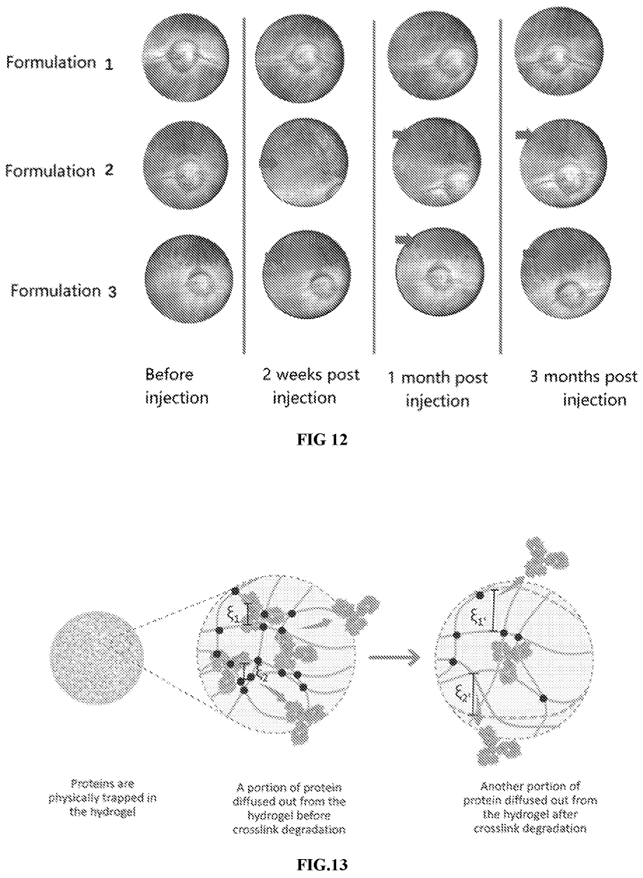

The injection of three forms of gel (Formulation 1, 2 and 3 of Table 8)) into rabbit eyes shows the gels are compatible to animal in short term and long term. No gross change in retinal structure or media clarity is seen for all three formulations (FIG. 12).

### Example 15 Degradation and Protein Release of Hydrogel Formed by HA-MI and DX-SH

15.1 Swelling StudyHA-MI of 27 kDa and 3% and 18% DM obtained in example 3 was dissolved in phosphate buffer (PB) at 120 mg/ml, wherein the HA-MI of 3% DM was dissolved in 0.02 M PBS, and the HA-MI of 18% DM was dissolved in 0.1M PBS. DEX-SH of 40 kDa and 5% DM was dissolved in PB at 240 mg/ml. After complete dissolution, both solutions were cooled downed in fridge for 15 min before mixing at 1:1 volume ratio (MI polymer/SH polymer mass ratio or MI/SH ratio=0.5). The formed gel was weighted for initial mass and then transferred into 2 ml of PBS containing 0.03% of sodium azide. The swelling study was performed in an incubator at 37° C. At each time point, the gel was blotted dried and weighted and the buffer was replaced with fresh PBS containing 0.03% of sodium azide.

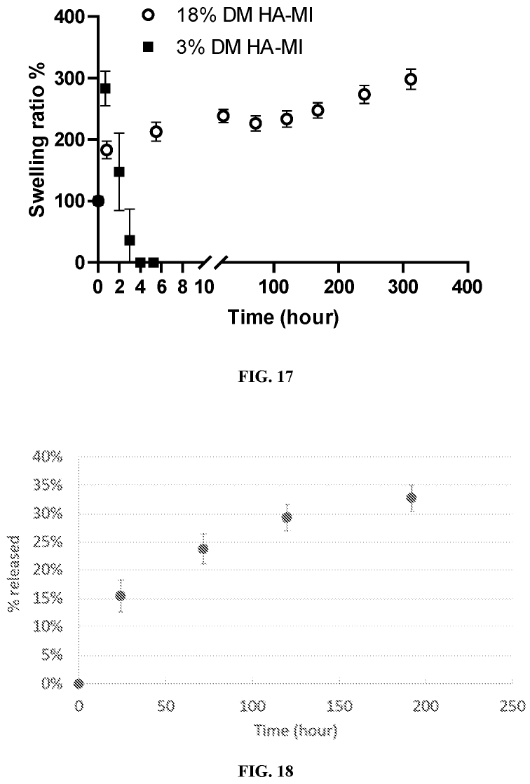

The results are shown in FIG. 17, which illustrates the polymer is degradable and the gel life of the hydrogel can be more than 300 hours.

15.2 Release Study

HA-MI of 27 kDa and 18% DM (8 mg) was dissolved in 67 μL of PBS. DEX-SH of 40 kDa and 6% DM (16.13 mg) was dissolved in 67 μL of Avastin. After complete dissolution, both solutions were cooled downed in an ice box for 15 minutes. On a piece of parafilm at room temperature, 20 μL of each HA-MI and DEX-SH solution was mixed (MI/SH ratio=0.5), and the gel was incubated at 37° C. for 30 minutes for gel formation. Afterwards, the gel was weighted and transferred to a centrifuge tube. PBS containing 0.03% of sodium azide was used as release buffer. At each time point, the buffer was taken out and replace with fresh buffer. The concentration of protein was measured by Bradford assay by manufacturer's instruction (Biorad).

The results are shown in FIG. 18, which illustrated that the protein was cumulatively released from said hydrogel in more than 200 hours.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A composition comprising a first hydrogel forming polymer and a second hydrogel forming polymer,
wherein the first hydrogel forming polymer is capable of reacting with the second hydrogel forming polymer to form a hydrogel, wherein the hydrogel is degradable and enables a sustained release of a target agent;
wherein
the first hydrogel forming polymer comprises a first hydrogel forming polymer derivative, the first hydrogel forming polymer derivative comprises a first modification, and the first hydrogel forming polymer derivative is electrophilic;
the second hydrogel forming polymer comprises a second hydrogel forming polymer derivative, the second hydrogel forming polymer derivative comprises a second modification, and the second hydrogel forming polymer derivative is nucleophilic; and
a mass ratio between the first hydrogel forming polymer and the second hydrogel forming polymer is less than 1.

2. The composition according to claim 1, wherein the first modification is at least one selected from the group consisting of a vinyl, an acryloyl, a thiol, an alkene, a thiolester, an isocyanate, an isothiocyanate, an alkyl halide, a sulfonyl halide, an epoxide, an imidoester, a fluorophenyl ester, a carbonate, a carbodiimide, a disulfide, and an aziridine.

3. The composition according to claim 1,
wherein the first modification is at least one selected from the group consisting of a vinylsulfone, a maleimide, an acrylate, a methacrylate, and an epoxide, and/or
the second modification is at least one selected from the group consisting of a thiol, an amine, an azide, a hydrazide, a diene, a hydrazine, and a hydroxylamine.

4. (canceled)

5. The composition according to claim 1, wherein the first hydrogel forming polymer and/or the second hydrogel forming polymer is at least one selected from the group consisting of a polysaccharide and a derivative thereof.

6. The composition according to claim 1, wherein the first hydrogel forming polymer and/or the second hydrogel forming polymer is at least one selected from the group consisting of a hyaluronic acid, a chitosan, a chondroitin sulfate, an alginate, a carboxymethylcellulose, a dextran, and a derivative thereof.

7. (canceled)

8. The composition according to claim 1, wherein the hydrogel is hydrolysable without involvement of degradative enzymes.

9. The composition according to claim 1, wherein at least one of the first hydrogel forming polymer and the second hydrogel forming polymer comprises a degradable linker.

10. The composition according to claim 9, wherein the degradable linker comprises a hydrolysable functional group.

11. The composition according to claim 10, wherein the hydrolysable functional group is at least one selected from the group consisting of an ester, an anhydride, and an amide.

12. (canceled)

13. The composition according to claim 1,
wherein the first hydrogel forming polymer derivative has a first average degree of modification (first DM) of less than about 40%, and
the second hydrogel forming polymer derivative has a second average degree of modification (second DM) of less than about 40%.

14. The composition according to claim 13, wherein a ratio between the first DM and the second DM is from about 3:1 to about 1:3.

15. The composition according to claim 1, wherein the first hydrogel forming polymer derivative is at least one of a dextran derivative modified with one or more vinylsulfone groups and a hyaluronic acid derivative modified with one or more vinylsulfone groups, and
the second hydrogel forming polymer derivative is at least one of a dextran derivative modified with one or more thiol groups and a hyaluronic acid derivative modified with one or more thiol groups.

16. (canceled)

17. The composition according to claim 1, wherein the composition is in the form of a powder.

18. The composition according to claim 1, wherein the composition is a liquid, and a concentration of the first hydrogel forming polymer and/or the second hydrogel forming polymer in the liquid is from about 1% w/v to about 50% w/v.

19. A hydrogel for a sustained release of a target agent, wherein the hydrogel is formed with the composition according to claim 1.

20. The hydrogel according to claim 19, wherein the hydrogel further comprises the target agent.

21. The hydrogel according to claim 19, wherein the target agent comprises a macromolecule.

22. (canceled)

23. The hydrogel according to claim 19, wherein at least about 20% of the target agent is a free target agent not conjugated to the hydrogel.

24. (canceled)

25. The hydrogel according to claim 19, wherein less than about 50% of the target agent is cumulatively released within an initial 24 hours from the hydrogel, and a remaining portion of the target agent is cumulatively released from the hydrogel in about 1 to about 36 months.

26. The hydrogel according to claim 19, comprising a macroscopic hydrogel and a micronized hydrogel.

27. (canceled)

28. A method for producing the hydrogel according to claim 19, comprising:
mixing the composition with a buffer to form a polymer solution; and
subjecting the polymer solution to a condition, wherein the condition enables formation of the hydrogel.

29. The method according to claim 28, wherein the subjecting of the polymer solution comprises injecting the polymer solution in a subject in need thereof.

30. The method according to claim 29, wherein the subjecting of the polymer solution further comprises incubating the composition at about 1° C. to about 45° C.

31. The method according to claim 28, wherein the polymer solution further comprises the target agent.

32-33. (canceled)

34. A method for a sustained release of a target agent, comprising enclosing the target agent in the hydrogel according to claim 19.

35. A kit, comprising:
a) the composition according to claim 1; and
b) a target agent to be sustained released by the hydrogel formed with the composition of a).

36-37. (canceled)
